(19) United States
(12) Patent Application Publication
(10) Pub. No.: US 2008/0039737 A1
(43) Pub. Date: Feb. 14, 2008

(54) Method and apparatus for measuring indices of brain activity during motivational and emotional function

(75) Inventors: Hans C. Breiter, Lincoln, MA (US); David Borsook, Concord, MA (US)

Correspondence Address: FISH & RICHARDSON PC, P.O. BOX 1022, MINNEAPOLIS, MN, 55440-1022, US

(73) Assignee: The General Hospital Corporation, a Massachusetts corporation

(21) Appl. No.: 11/807,535

(22) Filed: May 29, 2007

Related U.S. Application Data
(63) Continuation of application No. 09/729,665, filed on Dec. 4, 2000, now abandoned.
(60) Provisional application No. 60/168,660, filed on Dec. 2, 1999.
(60) Provisional application No. 60/193,300, filed on Mar. 30, 2000.
(60) Provisional application No. 60/228,950, filed on Aug. 28, 2000.

(51) Int. Cl. A61B 5/0484

(52) U.S. Cl. 600/544

(57) ABSTRACT

A method and apparatus for evaluating motivational and emotional circuitry in the brain during paradigms focused on specific motivational functions, to directly determine which components and how much the motivational and emotional brain circuitry responds. This circuitry response answers questions focused on normal and abnormal behavior, along with questions regarding the normal and abnormal function of the circuitry. The results of interrogating the motivational and emotional circuitry can be used for objectively measuring, in individual humans or animals, their preferences or responses to motivationally salient stimuli including but not limited to stimuli which are internal or external, conscious or non-conscious, pharmacological or non-pharmacological therapies, diseased based processes or not, financial or non-financial, etc. This method and apparatus for measuring brain activity during motivational and emotional functions can further be used to predict individual choices, preferences and planned behaviors, plus interpret internal experiences without recourse to the subjects participation or their voluntary description of these choices, preferences, planned behaviors, or internal experiences.

### RELATED APPLICATIONS

This application claims priority under 35 U.S.C. § 119(e) from U.S. provisional application Nos. 60/168,660 filed on Dec. 2, 1999, 60/193,300 filed on Mar. 30, 2000 and 60/228,950 filed on Aug. 28, 2000 all of which are hereby incorporated herein by reference in their entireties.

### GOVERNMENT RIGHTS

This work was sponsored by NIDA grants DA13296-01, DA00265 and DA09467. The government may have certain rights in this invention.

### FIELD OF THE INVENTION

This invention relates to non-invasive measurement methods and systems of and more particularly to method and apparatus for measuring indices of brain activity during motivational and emotional function

### BACKGROUND OF THE INVENTION

As is known in the art, magnetic resonance imaging (MRI) (also referred to as nuclear magnetic resonance or NMR) and other non-invasive techniques such as functional magnetic resonance imaging (fMRI), electroencephalogram (EEG), magnetoencephalography (MEG), positron emission tomography (PET), infrared imaging (IR), single photon emission computer tomography (SPE), computer tomography (CT) have been proposed to directly examine a combination of subcortical and cortical brain regions in humans which have been implicated by animal research in the fulfillment of motivational states. To date, however, this goal has not been accomplished. Many functional illnesses appear to involve some dysfunction of systems for motivation and emotion. Although this has not been proven, this hypothesis is supported by the commonality of symptoms and signs relating to motivation and emotion which are associated with many psychiatric disorders.

The brain systems which mediate motivation and emotion are complex, but significant progress has been made to begin dissecting the subsystems of motivation. Much of this effort has focused on investigations of “reward” systems, and depended on the use of functional imaging with experimental psychology. The central nervous system works on many spatial scales, though, so that brain function has to be investigated at multiple levels.

To investigate brain function at multiple scales, research needs to be performed with distinct methodologies to target these scales of brain function. The relationship of these research methods is such that many of these technologies can be combined to ask neuroscience questions at multiple levels of function. For instance, neuroimaging of animals is currently performed with animals that have depth electrodes a so-called “invasive” approach. The same experiment is carried out with functional imaging and electrophysiology, and the results then collated.

It would, however be desirable to provide a technique and system to non-invasively interrogate the brain of an individual regarding motivational states and decision making behavior (both conscious and unconscious) which fulfills these motivational states. It would be further desirable to define the brain circuitry mediating rewarding and averse functions with motivational states using a non-invasive measurement technique. It would also be desirable to define such motivationally relevant brain circuitry in individual subjects. It would be still further desirable to provide a non-invasive method and system which can objectively evaluate motivational states in individuals and allow predictions of current or future behavior to be made and to define past behaviors or mental states on the basis of measure activity in brain circuitry mediating rewarding and aversive function with motivational states.

### SUMMARY OF THE INVENTION

In accordance with the present invention, a system includes a non-invasive measurement apparatus for obtaining signals of central nervous system (CNS) activity, a localization processor, coupled to the non-invasive measurement system, for localizing signals to specific anatomical and functional brain regions, a correlator for correlating an experimental process to brain activity and a processor for interpreting the result of the correlation to a specific application.

With this particular arrangement, a system for measuring indices of brain activity during motivational and emotional function is provided. It should be appreciated that the non-invasive measurement apparatus may be provided as one which can implement fMRI, PET, IR, SPECT, CT, MRS, MEG and EEG or other techniques to non-invasively measure indices of brain activity during motivational and emotional function. The CNS signal processor and the correlation processor cooperate to determine indices of brain activity during motivational and emotional function. Suffice it here to say that once CNS signals are obtained the signals are localized to examine the function in a particular region of the brain. The particular manner in which such the signals are localized are dependent upon a variety of factors including but not limited to the technique or techniques (including equipment) used to extract the signals. Once signals are extracted, the correlation processor correlates empirical data with the measured signals and interprets the results of the correlation to a specific application. It should be appreciated that although the CND and correlation processors are described as separate and distinct processors, in practice the functions performed by these may be performed by a single processor or by more than one processor.

In accordance with a further aspect of the present invention a method for measuring indices of brain activity during motivational and emotional function includes the steps of non-invasively acquiring central nervous system (CNS) signals, statistically analyzing and then localizing the CNS signals to specific anatomical and functional brain regions, evaluating the CNS signals with regard to patterns of activity within and between functional brain regions, interpreting the results of the correlation to a specific application. With this particular arrangement, a technique for measuring indices of brain activity during motivational and emotional function is provided. In one embodiment, the CNS signals are acquired (e.g. via an MRI, PET system while the subject undergoes experimental paradigm focused on one or more “motivation/emotion processes. In other embodiments, the CNS signals are acquired while the subject is exposed to certain stimulus (e.g. the subject views photographs of people or food or consumer products) or while the subject performs particular tasks (e.g. presses a bar to get a particular result). Alternatively, the subject could perform some combination of the above tasks. A measuring apparatus which noninvasively obtains the CNS signals is used.

Data associated with the experimental/paradigm is correlated with patterns of activity and other measures. In one embodiment for example, brain responses in a region called the amygdala will be evaluated for habituation to aversion stimuli. If it does not habituate at or below a population normed average then individuals who are being tested with the diagnosis of obsessive compulsive disorder will not be referred for behavioral therapy since a common component of behavioral therapy is the ability to habituate or be de-conditioned to aversive stimuli.

In the step of interpreting the results of the correlation to a specific application, the subject's response to a known response for a particular application is made. For example, if a subject is being tested to determine whether or how much they like a particular product, the amount and/or intensity of activity in certain regions of the subjects brain is compared with signals from the subject's brain (or from a database of known brain region responses) in response to stimuli considered to be normal statistics for eliciting responses with a limited variance from the subject (e.g., extreme liking vs. extreme aversion). Based upon this information, a determination can be made as to whether or how much the subject liked the particular product.

### BRIEF DESCRIPTION OF THE DRAWINGS

The foregoing features of the invention, as well as the invention itself may be more fully understood from the following detailed description of the drawings, in which:

FIG. 1 is a flow diagram showing a general method for measuring indices of Central Nervous System activity during motivational and emotional function and determining indices of brain activity during motivational and emotional function;

FIG. 2A is a schema of Brain Functional Illness and its relationship to motivation/emotion function;

FIG. 2B is a schema detailing a category of brain functional illness (e.g., pain);

FIG. 2C is a generalized schema of motivational function, and dissection of one of its components;

FIG. 2D is a generalized schema which illustrates three phases of motivational function;

FIG. 3 is a block diagram of brain of brain circuitry of reward and aversive function and illustrates brain anatomy of reward and aversive function that is implicated in motivated behavior;

FIG. 3A is a graph showing a plot of signal strength from the left NAc vs. time for saline infusions;

FIG. 3B is a graph showing a plot of signal strength from the left NAc vs. time for morphine infusions;

FIG. 3C is a graph showing a plot of signal strength from the left and right NAc vs. time for morphine infusions;

FIG. 3D is a graph showing a plot of signal strength from the left and right NAc vs. time for saline infusions;

FIG. 3E, is a statistical activation map for significant signal change in the right nucleus accumbens;

FIG. 3F is a graph showing a plot of % signal strength change from the right nucleus accumbens vs. time;

FIG. 3G is a summary schematic of limbic and paralimbic brain regions observed in drug studies;

FIG. 3H, is a graph showing absolute fMRI signals for six regions of interest in reward regions vs. time;

FIG. 3I, is a graph showing absolute fMRI signals for four regions of interest in reward regions vs. time for three outcomes;

FIG. 3J is a graph of early reward circuitry activated to pain before subjective report of pain;

FIG. 3K shows activation of the SLEA during the early phase of a 46° C. stimulus;

FIG. 3L shows an activation map of the SLEA with no activation in the region during the late phase of a 46° C. stimulus;

FIG. 3M shows an activation map of the primary somatosensory cortex during the early phase of the stimulus;

FIG. 3N shows an activation map of the primary somatosensory cortex during the late phase of the stimulus;

FIG. 30 is a graph showing the time course of the signal in the primary somatosensory cortex;

FIG. 4 is a block diagram of a noninvasive measurement apparatus and system for measuring indices of brain activity during motivational and emotional function;

FIG. 5A is a flow diagram illustrating the general phases of a Motivational/Emotional Mapping Process (MEMP) according to the present invention;

FIGS. 5B-5C are a series of flow diagrams illustrating a MEMP schema for mapping motivational/emotional response; and

FIG. 6 is a diagram illustrating an association between functional neuroimaging in humans and animals. The importance of functional neuroimaging in humans and animals is apparent when considering that it is the primary means by which gene and molecular function can be linked to their behavioral effects.

### DESCRIPTION OF THE PREFERRED EMBODIMENTS

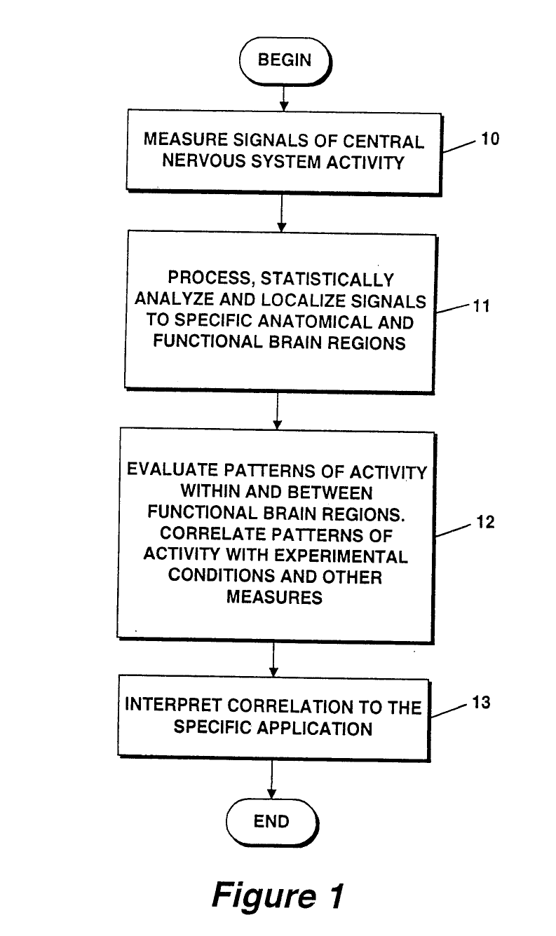

Referring now to FIG. 1, a flow diagram shows the processing to determine indices of Central Nervous System activity during motivational and emotional function. Such processing may be performed by a processing apparatus which may, for example, be provided as part of non-invasive measurement system such as that to be described below in conjunction with FIG. 4.

In the flow diagram of FIGS. 1 and 5A-5C, the rectangular elements in the flow diagrams are herein denoted “processing blocks” and represent computer software instructions or groups of instructions. The diamond shaped elements in the flow diagrams are herein denoted “decision blocks” and represent computer software instructions or groups of instructions which affect the processing of the processing blocks.

Alternatively, the processing blocks represent steps performed by functionally equivalent circuits such as a digital signal processor circuit or an application specific integrated circuit (ASIC). It should be appreciated that some of the steps described in the flow diagram may be implemented via computer software while others may be implemented in a different manner e.g. via an empirical procedure. The flow diagrams do not depict the syntax of any particular programming language. Rather, the flow diagrams illustrates the functional information one of ordinary skill in the art requires to fabricate circuits or to generate computer software to perform the processing required of the particular apparatus. It should be noted that many routine program elements, such as initialization of loops and variables and the use of temporary variables are not shown. It will be appreciated by those of ordinary skill in the art that unless otherwise indicated herein, the particular sequence of steps described is illustrative only and can be varied without departing from the spirit of the invention.

Turning now to FIG. 1, processing begins in step 10 in which after positioning subjects to be tested (e.g. persons who are under going a lie detection test) and instructing the subjects to remain as still as possible, Central Nervous System (CNS) signals are acquired. In one embodiment, the CNS signals are acquired while the subject undergoes experimental paradigm focussed on one or more “motivation/emotion processes. In other embodiments, the CNS signals are acquired while the subject is exposed to certain stimulus (e.g. the subject views photographs of people or food or consumer products) or while the subject performs particular tasks (e.g. presses a bar to get a particular result). Alternatively, the subject could perform two or more of the above tasks. A measuring apparatus which noninvasively obtains the CNS signals is used. In one embodiment, the subject to be tested is placed in a scanning region of an MRI or PET system of the type to be described below in conjunction with FIG. 4.

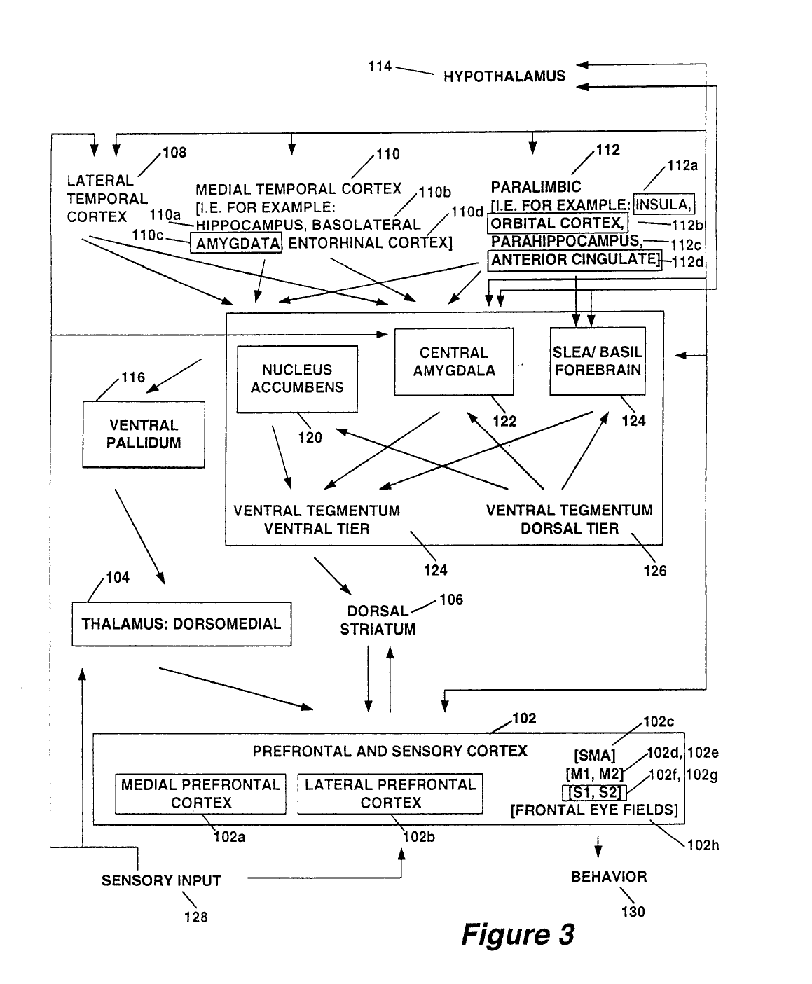

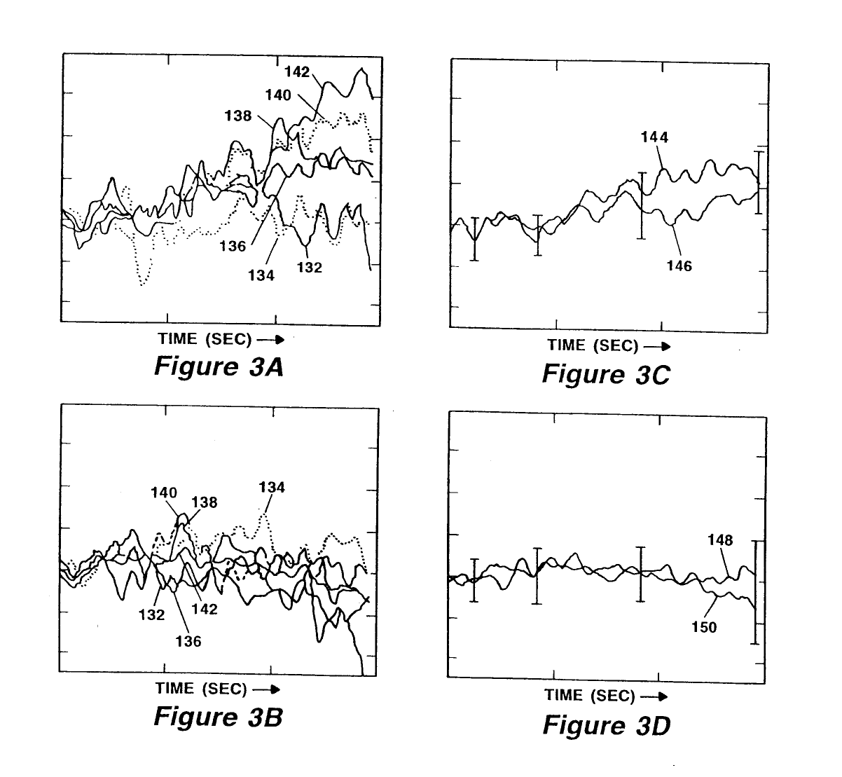

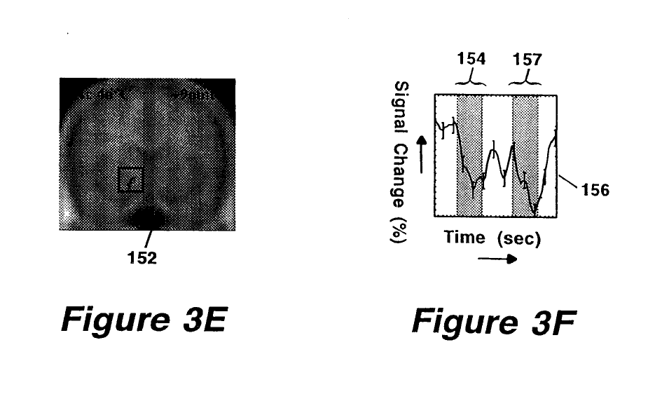

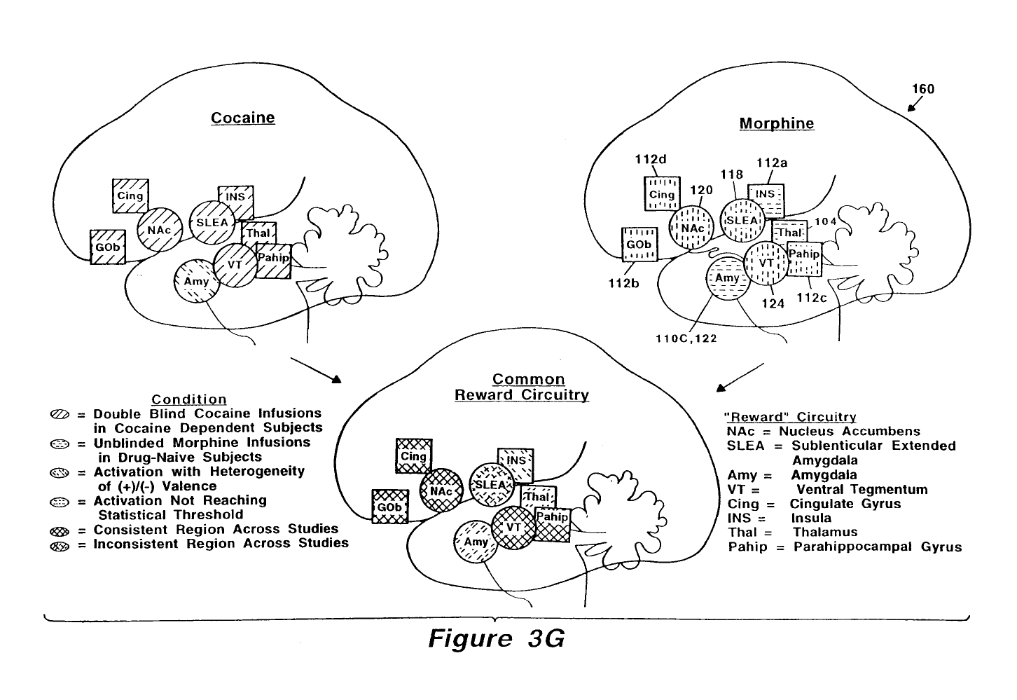

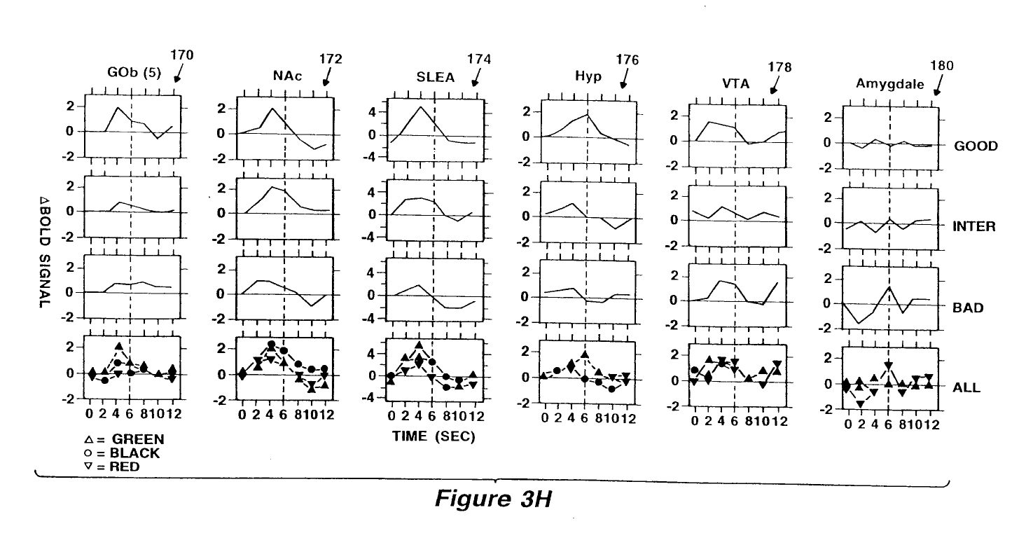

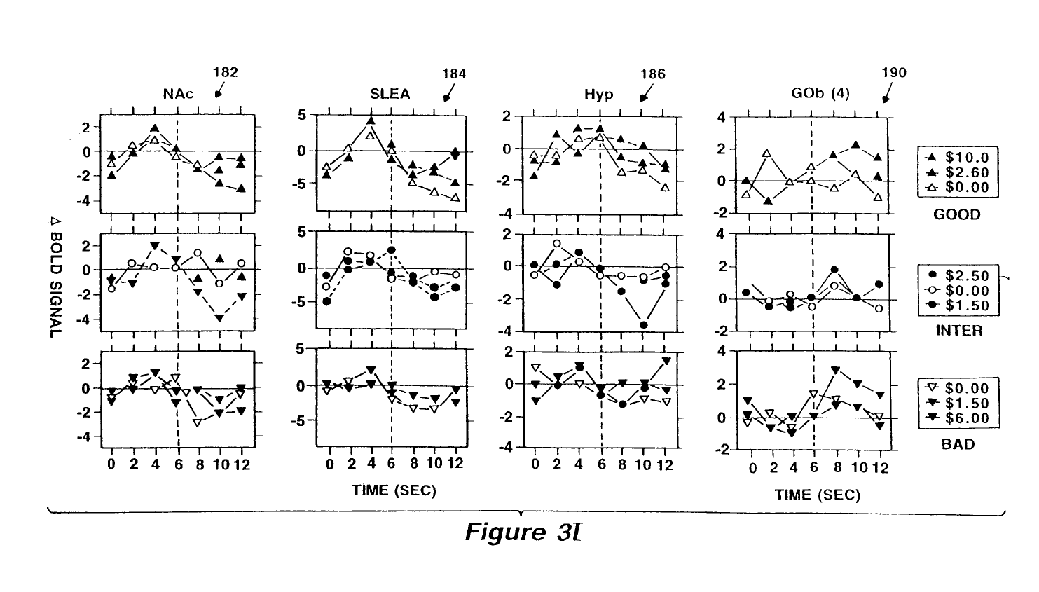

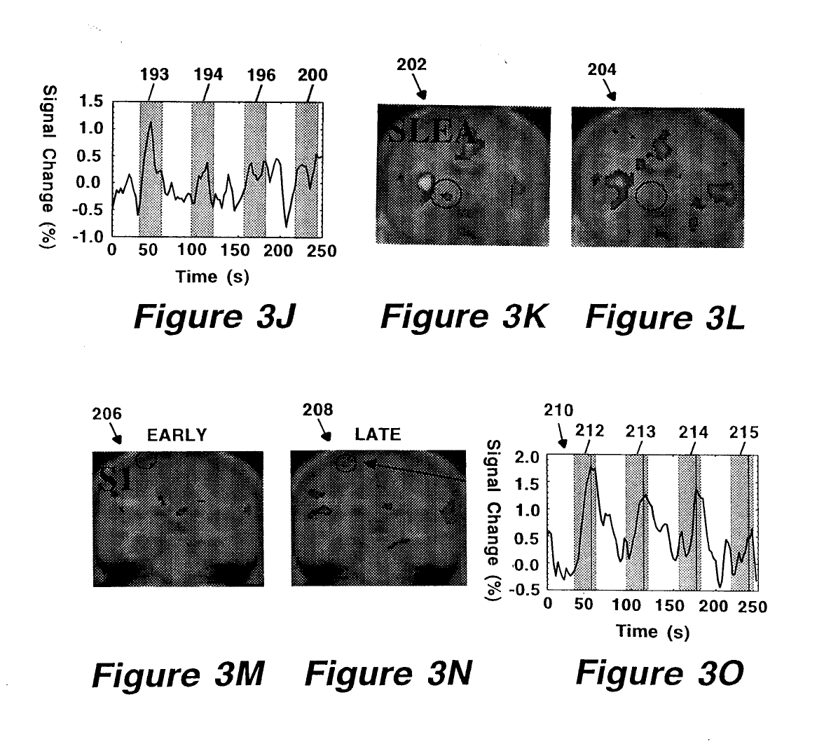

Processing then proceeds to Step 11 where the non-invasively obtained CNS signals are statistically analyzed and then localized to specific anatomical and functional brain regions. The details of this process are described below in conjunction with FIGS. 3-30 and 5A-5C.

Processing next proceeds to processing Step 12 where the experimental process CNS signals are evaluated with regard to patterns of activity within and between functional brain regions. Data associated with the experimental/paradigm is correlated with patterns of activity and other measures. In one embodiment, brain responses in a region called the amygdala will be evaluated for habituation to aversion stimuli. If it does not habituate at or below a population normed average then individuals who are being tested with the diagnosis of obsessive compulsive disorder will not be referred for behavioral therapy since a common component of behavioral therapy is the ability to habituate or be de-conditioned to aversive stimuli.

In process Step 13, an interpretation of the correlation obtained in Step 12 to a specific application is then made. In this step, the subject's response to a known response for a particular application is made. For example, if a subject is being tested to determine whether or how much they like a particular product, the amount and/or intensity of activity in certain regions of the subjects brain is compared with signals from the subject's brain (or from a database of known brain region responses) in response to stimuli considered to be normal statistics for eliciting responses with a limited variance from the subject (e.g., extreme liking vs. extreme aversion). Based upon this information, a determination can be made as to whether or how much the subject liked the particular product.

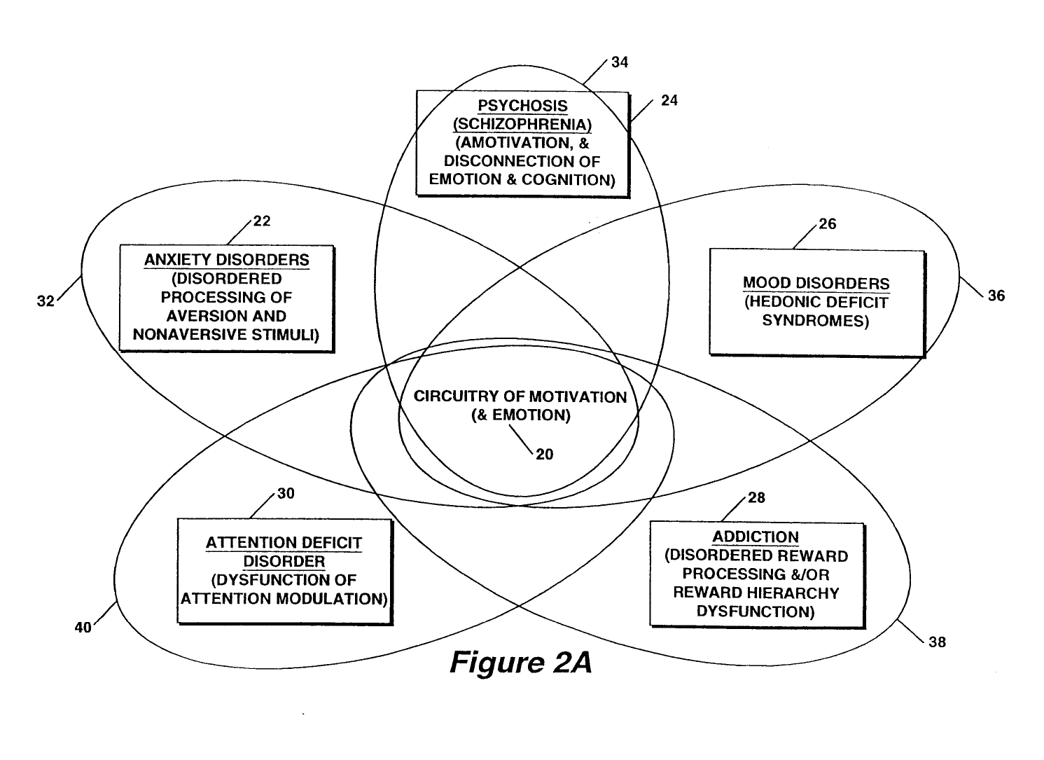

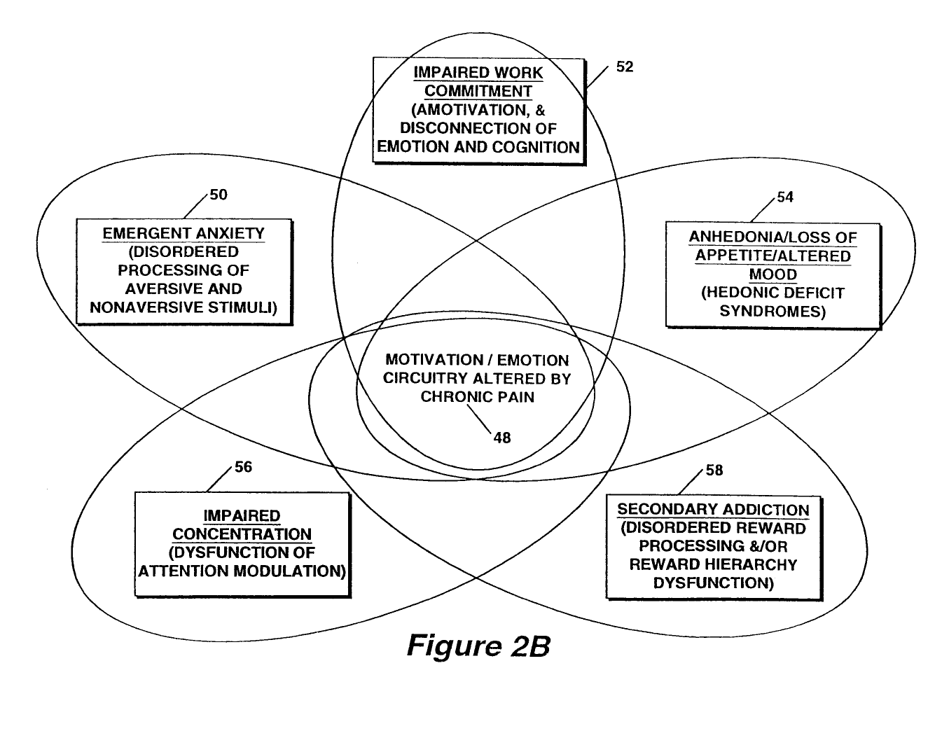

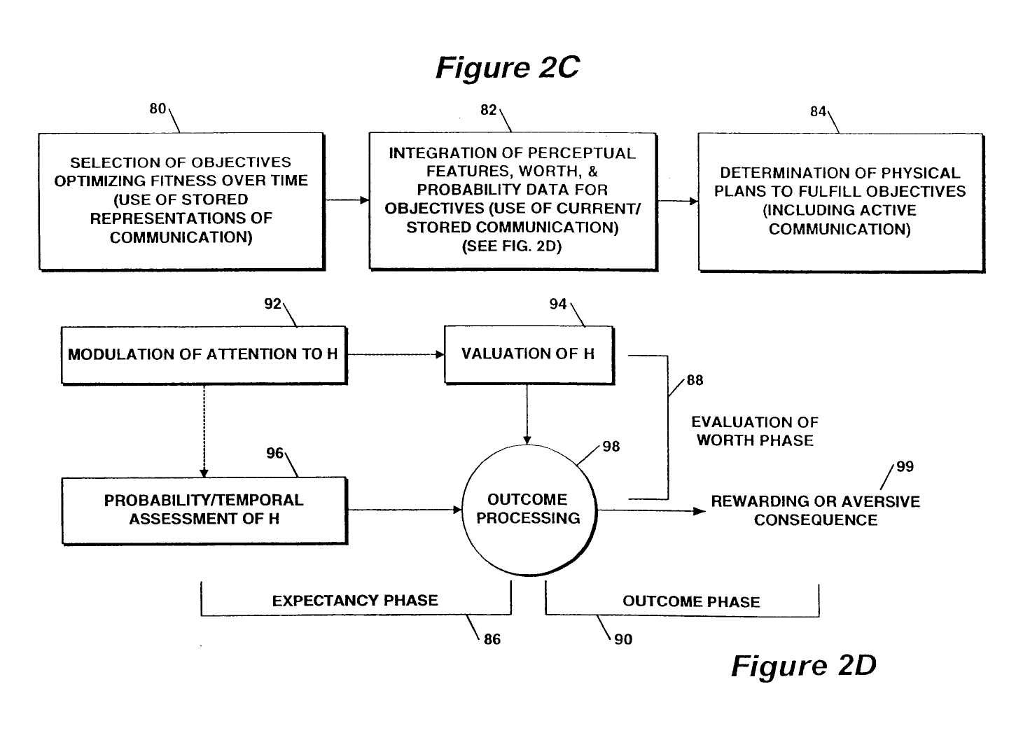

FIG. 2A is a schema of Brain Functional Illness and its relationship to motivation/emotion function. Psychiatric illnesses, pain disorders, and illnesses producing neuropsychiatric dysfunction are examples of brain functional illnesses. At the core of all psychiatric illness, is some disorder of motivation/emotion dysfunction. This has been most closely evaluated for substance abuse/addiction. The schema of FIG. 2A shows that relationships between circuitry of motivation 20 and a plurality of different categories of disorders designated by reference numbers 22-30 exists. Oval shaped reference lines 32-40 indicate that relationships exist between each of the disorder categories 32-40 and the circuitry of motivation and emotion 20. The details of the circuitry of motivation and emotion 20 are described in conjunction with FIGS. 3-5C below.

Referring now to FIG. 2B, a chart or schema which shows the relationship between circuitry of motivation altered by chronic pain 48 and a plurality of different behavioral states 50-58. Reference lines 62-70 indicate that relationships exist between each of the behavioral states 50-58 and the circuitry of motivation and emotion 48. It should be understood that pain is not traditionally considered a psychiatric disorder. Rather, pain is considered to be a functional illness. Thus, FIG. 2B is a schema detailing a category of brain functional illness (i.e., pain). Long term behavioral manifestations of pain include a constellation of symptoms aside from pain intensity, which closely parallel symptoms related to motivation and emotion observed with psychiatric illness. Thus, a close similarity exists between FIGS. 2A and 2B.

Referring now to FIGS. 2C and 2D, schema of motivational function are shown. As shown in FIG. 2C, motivated behavior necessitates at least three fundamental operations. These operations include: (1) selection of short-term and long-term objectives focused on attaining rewarding outcomes while avoiding aversive outcomes as shown in block 80, (2) integration of perceptual features regarding the rate, delay, incidence, intensity, (i.e., worth), amount, and category of these potential outcomes as shown in block 82, and (3) determination of physical plans involving musculature or organ function to obtain these outcomes as shown in block 84.

A simplistic rendition of subsystems needed for pulling H (where H corresponds to information as conceived and defined by Shannon & Weaver which is hereby incorporated herein by reference in its entirety) from the environment regarding potential rewards and aversive outcomes might segregate a subsystem for modulation of attention to putative goal-object features, a subsystem for probability assessment, and a subsystem for valuation. In congruence with prospect theory, probability computations would be processed in parallel with computations assessing value to determine the reward outcome as shown in FIG. 2D.

FIG. 2D illustrates three phases: (a) an expectancy phase 86; (b) an evaluation of worth phase 88; and (c) an outcome phase 90. If one considers variables needed to determine worth, one fundamental variable is the “rareness” of the goal-object in the environment, while a second is the value of the goal-object to the organism for reducing an existing “deficit state”. The former variable of “rareness” depends on a probability assessment for its computation, and thus is an important input to any function of worth evaluation.

The integration of perceptual features regarding the rate, delay, incidence, intensity, amount, and category of these potential outcomes as shown in block 82 can be represented as shown in blocks 92-98 of FIG. 2D. In block 86, modulation of attention to h refers to the increased attention a subject gives to the source of information “H.” This increased attention leads to “valuation of H” as shown in block 94.

FIG. 3 is a block diagram of brain circuitry 100 corresponding to brain circuitry of reward and aversive function. That is, FIG. 3 shows the route by which the brain receives sensory information and how that information propagates to various regions of the brain to produce motivated behavior. It should thus be appreciated that circuitry 100 illustrates brain anatomy of reward and aversive function that is implicated in motivated behavior.

The brain circuitry 100 includes a prefrontal and sensory cortex 102 which includes a medial prefrontal cortex 102a and a lateral prefrontal cortex 102b. The region 102 also includes the primary sensory motor components primary sensory/motor components 102c-102h relating to the behavior of the organism include regions such as the primary somatosensoy cortex S1 102f, the secondary sensory cortex S2 102g, the primary motor cortices (M1) 102d, and secondary motor cortices (M2) 102e which are involved in executing motor behavior. Planning of motor behavior includes regions such as the supplementary motor cortex (SMA) 102c. The frontal eye fields (102h) controls motor aspects of eye control relating to directing the reception of visual signals from the environment to the brain (It should be understood that signals are initially received by the primary and secondary visual cortices).

Brain circuitry 100 also includes the dorsomedial thalamus region 104, the dorsal striatum region 106 and the lateral and medial temporal cortex regions 108, 110. The medial temporal cortex region 110 includes, for example, the hippocampus 110a, the basolateral amygdala 110b, and the entorhinal cortex 110. Also included as part of the brain circuitry 100 is the paralimbic 112 which includes, for example, the insula 112a, the orbital cortex 112b, the parahippocampus 112c and the anterior cingulate 112d. Lastly the brain circuitry includes the hypothalamus 114, the ventral pallidum 116 and a plurality of regions collectively designated 118.

The regions collectively designated 118 comprises the nucleus accumbens (NA_{c}) 120, the central amygdala 122, the sublenticular extended amygdala of the basal forebrain SLEA/basal forebrain or SLEA/BF) 124, the ventral tegmentum (ventral tier) 126 and the ventral tegmentum (dorsal tier) 126.

The regions 118 collectively represent a number of regions having significant involvement in motivational and emotional processing. It should be appreciated that other components such as the amygdala 110b and 110c, are also important but not included in the regions designated by reference number 118. Other regions that are also important to this type of processing include the hypothalamus (114), the orbitofrontal cortex (112b), the insula (112a) and the anterior cingulate cortex (112d). Further regions are also important but listed separately such as the ventral pallidum (116), the thalamus (104), the dorsal striatum (106), the hippocampus (110a), medial prefrontal cortex (102a), and lateral prefrontal cortex (102b). Not listed in this figure but also involved in processing sensory information for its emotional implications is the cerebellum.

The specific functional contribution of each of these major regions are listed below. It should be noted that what follows is a gross simplification and does not convey the complexity nor the diversity of the functions that these regions have been implicated with and may in the future be connected to. Further note that there is currently a debate regarding the modular vs. non-modular function of these brain regions, i.e., can a specific function be attributed to each region in isolation. Accordingly what is listed below is information which provides on of ordinary skill in the art with the understanding that this function may be mediated by the connection with this region with many other regions (i.e., distributed function).

As a brain region the NAc has previously been implicated in the processing of rewarding/addicting stimuli, and is thought to have a number of functions with regard to probability assessments and reward evaluation—It has also has been implicated in the moment by moment modulation of behavior (e.g., initiation of behavior).

The SLEA/BF has been implicated in reward evaluation, based on its likely role in brain stimulation reward effects. It is thought to be important for estimating the intensity of a reward value. It and other sections of the basal forebrain appear to be important for the processing of emotional stimuli in general, and it has been implicated in drug addiction.

Like the NAc, the amygdala has been implicated in both processing of emotional information along with processing of pain and analgesia information. The amygdala has been implicated in both the orienting to and the memory of motivationally salient stimuli across the entire spectrum from aversion to reward. It may be important for the processing of signals with social salience in real time. In this context it is often referred to with regard to fear. A number of its anatomical connections to primary sensory cortices, suggest that it is important for the modulation for attention to motivationally salient stimuli.

With respect to the VT/PAG, Doparminnergic projections are present from the VT to the SLEA, the orbitofrontal cortex the amygdala and the NAc. Indeed dopaminergic projections go to most subcortical and prefrontal sites. The VT has been implicated in reward prediction processes, motor and a number of learning processes around motivational events in general. The PAG has also been implicated as a modulator of pain stimuli, for example, and may therefore be a region that signals early information on rewarding or aversive stimuli.

The Gob component of the prefrontal cortex has been implicated in a number of cognitive, memory, and planning functions around emotional stimuli or regarding rewarding or aversive outcomes in animal and human studies. This section of the prefrontal cortex has also been implicated in modulating pain. It has afferent and efferent connections with a number of subcortical structures including NAc and the VT. The GOb is involved in a number of different reward processes including those of expectancy determination and valuation. Patients with lesions in this region have impulse control problems.

The hypothalamus is involved in the monitoring and maintenance of homeostatic systems (e.g., endocrine control, satiety, thermoregulation, thirst monitoring, reproductive control, and pain processing). It also has been both implicated in the evaluation of the relevance for rewarding and aversive stimuli in order to maintain homeostatic equilibrium. The hypothalamus is highly important for meeting the objectives which optimize fitness over time and meet the requirements necessary for survival.

The cingulate cortex has been interpreted to be involved in attention and planning, the processing of pain unpleasantness the processing of reward events and emotions in general, and the evaluation of emotional conflict. The cingulate cortex is an extensive region of brain cortex and appears to have emotional and cognitive subdivisions to name a few.

The insula has been implicated in number of functions including the processing of emotional stimuli, the processing of somatosensory functions (e.g., pain), and the processing of visceral function.

The thalamus is composed of a number of sub-nuclei which have been implicated in a diverse range or functions. Fundamental among these functions appears to be that of being an informational relay of sensory and other information between the external and internal environment. It has also been directly implicated in both rewarding and aversive processes and damage to the structure may result in dysfunction such as chronic pain.

The hippocampus has been extensively implicated in functions for encoding and retrieval of information. Lesions to this structure lead to severe impairment in the ability to form new memories. Motivated behavior is heavily dependent on such memories: for instance, how a particular behavior in the past led to obtaining a goal object which would reduce a particular deficit state such as thirst or addictive behaviors.

The ventral pallidum region is one of the primary output sources of the NAc and has a number of projection sites including the dorsomedial nucleus of the thalamus. Via this connection it is one of the major relays between the NAc and the rest of the brain, in particular prefrontal cortical regions. It has been strongly implicated in reward functions and is a site thought to be important for the development of addiction.

The Medial Prefrontal Cortex region of the brain has been strongly implicated in reward functions and has been found to be one of the few brain sites into which cocaine self administration can be initiated in animals. There is strong data linking this region to attentional functions which are stressful or at the service of various motivational states.

In response to reward and aversion situations, certain regions of the brain circuitry 100 play a role in determining a response or action as discussed above. These regions are designated reward and aversion regions of the brain circuit. The activation of such reward and aversion regions can be observed during positive and negative reinforcement using neuroimaging technology. These reward and aversion regions produce specific functional contributions to motivated behavior. For example, contributions made by regions such as the nucleus accumbens (NAc) include assessment of probability (i.e. expectancy).

Central to performing valuation, probability assessment, and other information processing tasks needed for planning behavior in response to reward and aversion situations are a number of core brain regions including the nucleus accumbens (NAc) 120, the sublenticular extended amygdala of the basal forebrain (SLEA/BF) 124, amygdala (multiple nuclei) 110c, 122, the ventral tegmentum/periaqueductal gray (VT/PAG) 124, 126, the hypothalamus 114 and the orbirtal gyrus (GOb). The GOb is designated as the orbital cortex 112b in FIG. 3. Also important to reward and aversion information processing are regions such as the insula 112a, anterior cingulated 112d, thalamus 104, ventral pallidum 116, medial prefrontal cortex 102a, and cerebellum (not shown in FIG. 3). The cerebellum is associated with integrating motor and autonomic behavior. It appears to have specific roles in reward and emotion, including the detection of errors in information processing or the implementations of motor behaviors.

As shown on FIG. 3, when a subject receives or senses an input 128, the sensory input is generally processed by the brain circuitry in the following manner. The sensory input is sensed by the pre-fontal and sensory cortex, 102, the dorsomedial thalamus region 104 and the lateral temporal cortex 108. Signals are passed between the dorsomedial thalamus region 104 and the pre-fontal and sensory cortex 102. Signals are also passed between the pre-fontal and sensory cortex 102 and the dorsal striatum 106. The sensory input signals provided to the lateral temporal cortex 108 are passed to the region 118 and in particular to the nucleus accumbens 120 and the central amygdala 122.

Signals are also passed between the prefrontal and sensory cortex 102 and the region 118 (and in particular to regions 122, 124) as well as the hypothalamus 114. Interaction between the region 118 and the lateral temporal cortex 108, the medial temporal cortex 110 the paralimbic 112. Each of these interactions cause the regions to produce specific functional contributions to motivated behavior which is manifested as indicated at 130.

Referring now to FIGS. 3A-3D, in one experiment, core brain regions implicated in reward and aversive function were observed to activate in cocaine addicts after cocaine administration. In that experiment, the cocaine was administered after a brief abstinence from the drug in a randomized double-blind fashion relative to saline. Significant signal change was observed for the NAc 120 and SLEA regions 118 following cocaine with distinct time courses that correlated with subjective reports made by the subjects. Subjective reports of rush and craving from cocaine were correlated with distinct sets of brain regions activated. In particular, the NAc 120 and amygdala 110c, 122 were correlated with the motivational state of craving, while areas such as the SLEA/BF 118 and VT 124, 126 were correlated with the rush produced by cocaine.

The curves shown in FIGS. 3A-3D illustrate that activation of reward regions such as the NAc 20 after low dose morphine in healthy volunteers (as opposed to addicts) can be observed and illustrate signal changes in the Nac 120 observed in individuals over a period of time. FIGS. 3A-3D thus demonstrate the power of neuroimaging to interrogate reward and aversion circuitry in individuals even with mild perturbations.

Turning now to FIGS. 3A and 3B, plots of signal strength vs. time are shown. Time-course data (i.e. curves 132-142) from the left NAc in five subjects are shown for both morphine and saline infusions (FIGS. 3A, 3B respectively). Percent signal change in FIGS. 3A and 3B are normalized relative to each subjects pre-infusion baseline, but not detrended. The average signal change for the five subjects is shown as a black line, and the average infusion interval, given cardiac-gating of the acquisition, is shown as a blue bar below the fMRI signal intensity. The time-course data was sampled from each individual using a region of interest from the aggregate statistical map with each voxel localized in NAc meeting probability a threshold of p<0.05.

FIGS. 3A, 3B show that individual signals can be readily obtained in these small motivationally relevant regions. It also shows that there is a congruence of positive signal for a rewarding stimulus for this particular region (as opposed to a congruence of data for negative signal changes from other motivationally salient stimuli for this region.

Referring now to FIGS. 3C and 3D, individual time-course data after morphine and after saline is averaged separately for the right (curve 146—morphine: curve 148—saline) and left (curve 144—morphine: 150—saline) NAc. Error bars are included for the MRI data acquired as the 20′ time-point, the 70′ time-point, the 150′ time-point, and the 250′ time-point. Time is represented in seconds using a conversion of repetition time JR)=6 RR intervals=6 seconds. These graphs show that there were bilateral NAc changes to this particular rewarding stimulus, which is not always the case as noted in the summary figure for multiple reward experiments.

Referring now to FIG. 3E, the statistical activation map for significant signal change in the right nucleus accumbens (152), averaged for 6 subjects is shown.

Referring now to FIG. 3F, the average time course 156 (i.e., % signal change vs. time) of the activation shown in FIG. 3E for the same six subjects is shown. Note the correlation between the change in signal and the duration of the painful thermal stimuli (46° C.) shown as dark bars, Note that the signal goes down during the periods 154 and 157 in which the painful thermal stimulus is applied, it returns toward baseline during the inter-stimulus interval (i.e., between offset of 154 and onset of 157) and goes negative again during the second application of the thermal stimulus (157) The decrease in signal is highly significant because it shows that an aversive stimulus is negatively valenced (i.e. has a signal change opposite to that of rewarding stimuli).

Referring now to FIG. 3G, reward and aversion regions activated for both cocaine in addicts, and morphine in healthy volunteers, are juxtaposed to demonstrate the commonality of this circuitry.

FIG. 3G thus corresponds to a summary schematic of limbic and paralimbic brain regions observed with double blind cocaine infusions in cocaine dependent subjects (in purple and yellow), and unblinded low-dose morphine infusions in drug-naive subjects (in orange and blue). Regions activated to a significant degree in each study and not associated with heterogeneity of activation valence (i.e.—, positive vs. negative signal changes), are summarized in the brain schematic at the bottom of the image (in pink and green). Regions symbolized by a circle are sub-cortical regions traditionally associated with reward function in animal studies, while regions symbolized with squares are those associated in humans with emotion function in general. The commonality of activation across two distinct categories of drugs, in the NAc (120), SLEA (118), VT (124), and amygdala (110c 122) along with regions such as the cingulate cortex (112d) and orbital cortex—GOb (112b), suggest that a broad set of brain regions may be involved with generalized reward functions. Other regions included in the figure are the insula (112a), the thalamus (104) which is involved in sensory and motor integration and transmission and the parahippocampal gyrus (112c) which is involved in processing facial and location features. This shows that there is a generalized circuit of reward that responds to divergently different categories of drug.

Referring now to FIG. 3H, absolute fMRI signals are displayed for six regions of interest in reward regions. Signals were zeroed relative to the 8 second pre-stimulus epoch. The time-courses for the good (green), intermediate (black), and bad (red) spinners are displayed against gray-tone with the 95% confidence intervals in white. The dashed lines segregate the expectancy and outcome phases of the experiment. The bottom graphs illustrate the good, intermediate, and bad spinner time-courses together, using the same color-coding as in the columns of signals above them. The five columns of GOb(5) (170), NAc (172), SLEA (174), Hyp (176) and VT (178) signal represent signals with strong good spinner effects during the expectancy phase of the experiment. In the left amygdala (180) focus shows minimal effect during the good and intermediate spinners, and strong biphasic effects during the bad spinner. Differential responses to discrete expectancy conditions are shown for the five other reward regions including the NAc, SLEA. Hypothalamus, VT and GOb. This is the first demonstration of controlled expectancy effects in humans and further shows that the waveforms in each of these regions were significantly different. This data provides evidence that probability functions are computed by distributed sets of reward regions.

Referring now to FIG. 3I, the robust time-courses for bin effects in four ROIs are illustrated. Bins on the good spinner are shown in the top row of graphs, while bins for the intermediate spinner are shown in the middle row, and bins for the bad spinner are shown in the bottom row. The 8 seconds of data acquired before the outcome phase of the experiment are used to zero the data. The three columns of data from the NAc (182), SLEA (184), and Hyp (186) in (a) are grouped to illustrate regions that show differential effects for predominant gains as outcomes in the context of good expectancy. It should be noted that these three ROIs show differential effects for the outcomes on the good spinner. And demonstrate strict ordering on the basis of outcome magnitude. Similar orderings are not observed for outcomes in the context of intermediate and bad expectancies. These orderings are salient for supporting the notion that a distributed set of human brain regions represents stimulus worth, in a parametric fashion. The GOB (190) is presented to illustrate a very different profile of outcome responses. Namely, this ROI appears to respond to extremes, such as the $10.00 outcome in the context of good expectancy, and the −$6.00 outcome in the context of bad expectancy. Differential responses to discrete monetary outcomes in a number of reward regions in particular this finding demonstrates that magnitude differences in the valuation of rewarding stimuli can be distinguished. This shows that reward functions are not dust “on” “off’ phenomena but produce a gradation of response across the continuum of reinforcement (i.e., between reward and aversion). These data indicate that the brain can discriminate nuances in reward—value—Such observations show that a mechanism exists for determining what an organism values, and the relationship of this valuation to valuation of other objects, events, or internal states.

Referring now to FIGS. 3J-30, early reward circuitry activated to pain before subjective report of pain.

Referring now to FIG. 3I, the graph shows the time course of the signal (% change vs. time) for activation in the SLEA following a 46″C stimulus—Note that there is a large initial change in the signal (192) during the first epoch 193 of the thermal stimulus and not during subsequent thermal epochs (194, 196, 200).

Referring now to FIGS. 3K and 3L, these figures shows activation in the SLEA (a putative reward structure) during the early (202) and no activation in the region during the late (204) phase of a 46° C. stimulus. Other activations in the figure represent known regions including the right and left insula (112—in FIG. 3) and the cingulate gyrus (112d—in FIG. 3).

Referring now to FIGS. 3M and 3N. The figures show relatively little activation in the primary somatosensory cortex (S1) 102f (FIG. 3) (206) during the early phase of the stimulus while there is significant activation during the late phase of the stimulus (208) in the corresponding region. Other areas of activation include the insula (112—in FIG. 3).

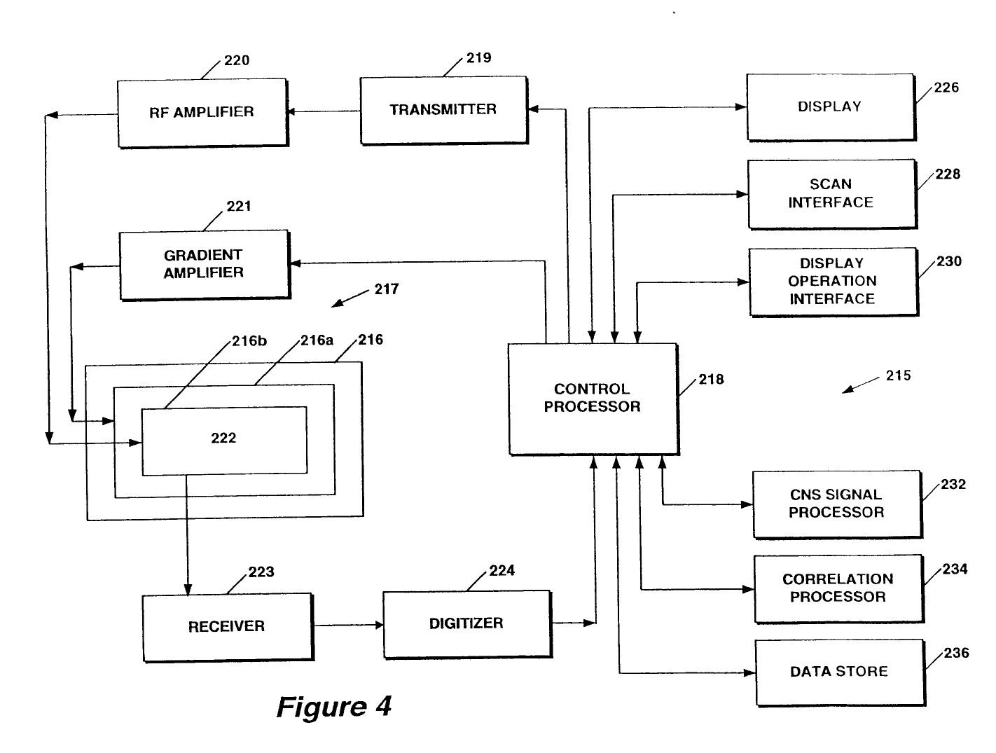

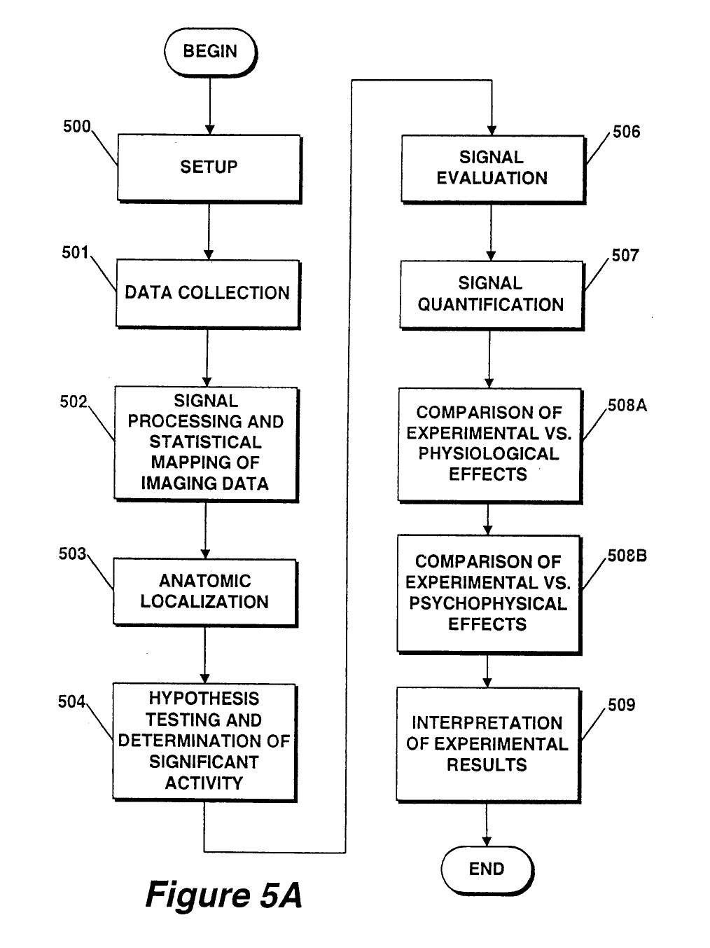

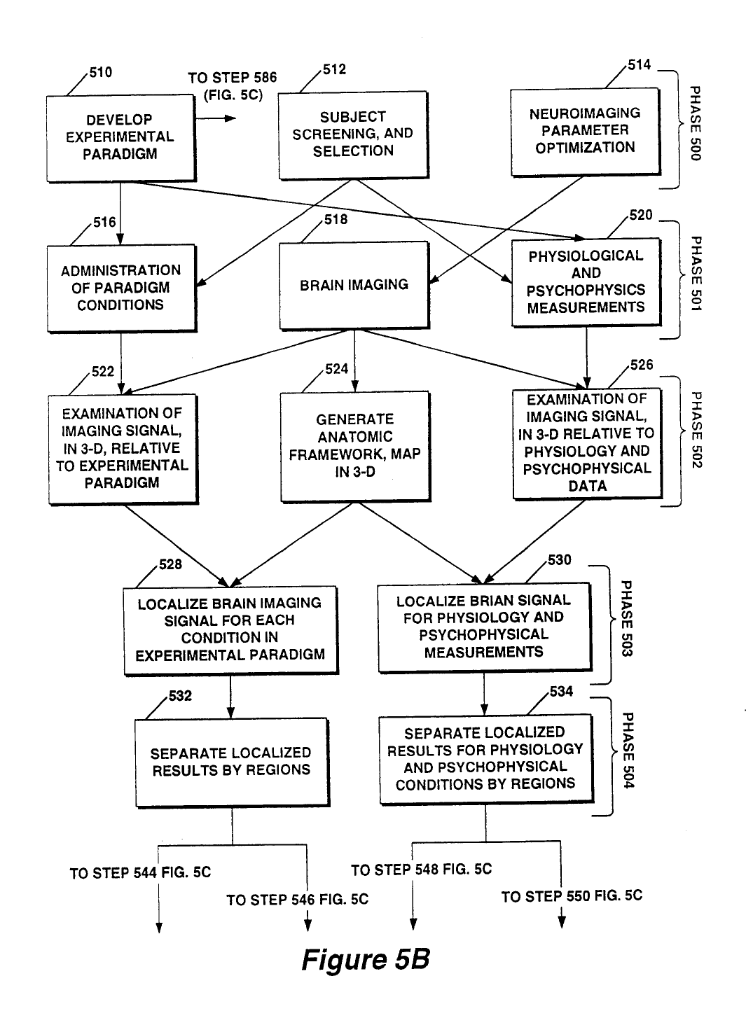

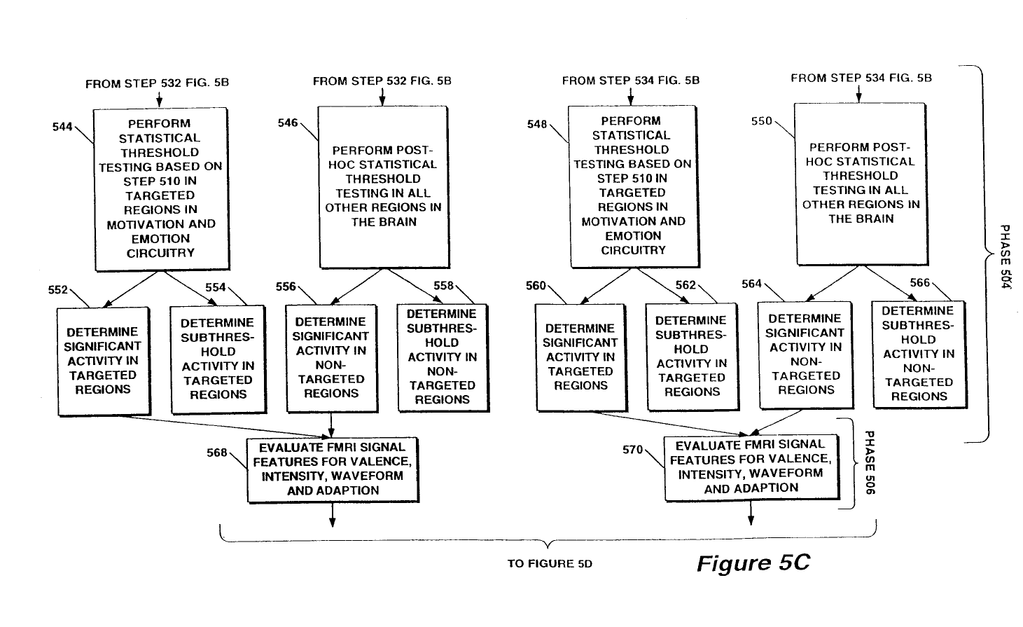

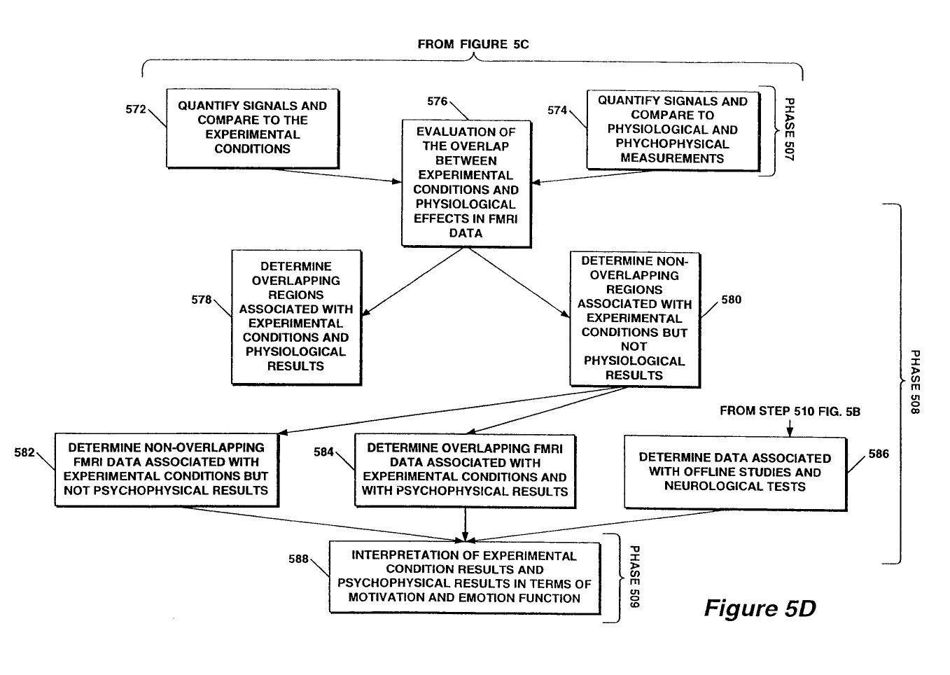

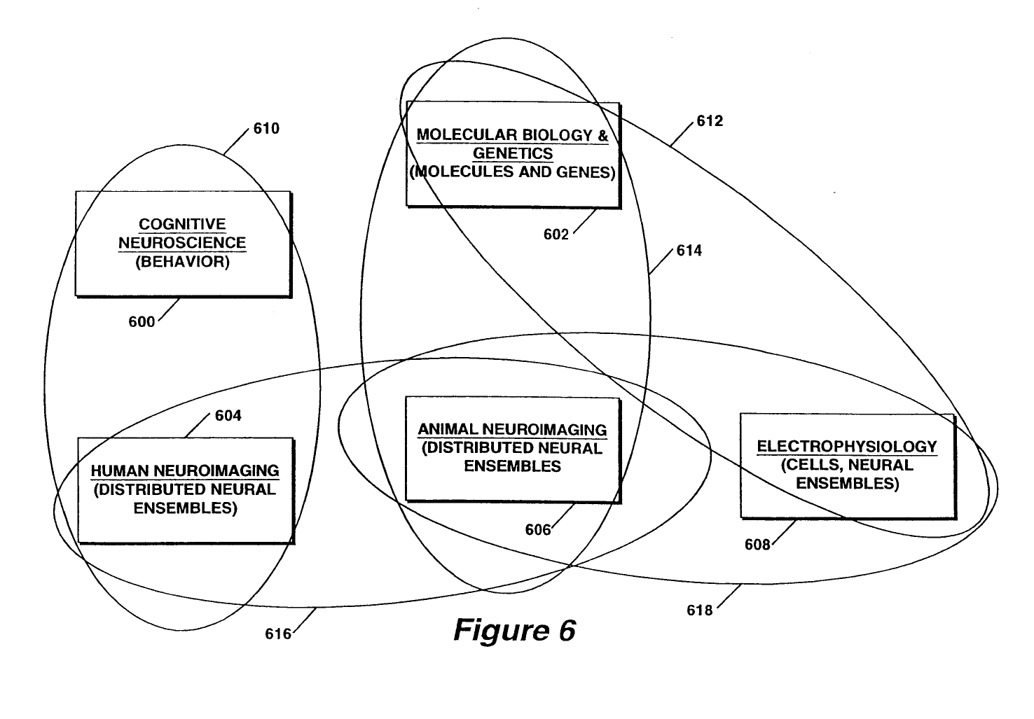

Referring now to FIG. 30, the graph shows activation (210) or time course of the signal in the primary somatosensory cortex 102 (FIG. 3). It should be noted that activation exists in each of the time periods 212-215 during which the thermal stimulus is applied (each time period referred to as an epoch).

It should also appreciated that FIGS. 3J-30 show why regions such as the SLEA, which has been heavily implicated in reward valuation respond to an aversive stimulus ahead of systems involved with primary somatosensory perception. The SLEA response occurred before the subjects made conscious ratings that they were feeling pain. This is an example of how neuroimaging can be used to differentiate conscious from non-conscious processes with relevance to motivation.

It should be appreciated that distinct patterns of reward and aversive circuitry function can be observed after presentation of different valences of stimuli (i.e., fearful vs. happy or neutral faces) to different subjects. It is important to note, for example, that both happy and fearful signal habituates rapidly over the course of an experiment. This indicates that the brain-adapts to novel emotional information quickly and that the techniques of the present invention can be used to observe this function.

It has been observed that right amygdala activation occurs after a different category of aversive stimulus (i.e., sad faces). It should also be appreciated that demographic differences in subjects can lead to different activation in different groups of subjects (e.g. male vs. female) to the same stimulus. For example, nucleus accumbens and amygdala activation to fearful faces are different in groups of men and women.

Demographic differences in subjects can lead to different activation in different groups of subjects (e.g. male vs. female) to the same stimulus. For example, distinct differences in activation of reward-relevant regions between men and women, particularly for the mid-luteal phase of the menstrual cycle have been found.

Also, drug expectancy effects can be observed prior to the infusion of cocaine vs. saline. For example, NAc activation can be observed prior to and shortly after infusions, but before the onset of any pharmacological effects. These effects result from probability assessments regarding the potential of receiving a drug reward (i.e. a previously experienced reward). This demonstrates that subsystems of motivational circuitry function can be interrogated in isolation of other subsystems. In addition, subjects did not intend to signal their expectancy of drug, yet the neuroimaging technology recorded it.

In one experiment, a game of chance (similar to gambling) was used. In this experiment, a wheel of fortune having a spinning arrow on it was used. The spinning wheel lands to signal the reception of a reward (money). This gives an example of the type of experiment that can be done for almost any demographic group. In such an experiment, expectancy (predicted chance of winning) and outcome (actual winning or dollars earned) processes are segregated in time.

In the experiment, subjects have the opportunity to lose money as well as win money since spinners are randomly presented in this experiment. The overall sequence of potential winnings and losses resembles a random walk process like that of a stock index. In one particular case, the overall trend was positive because gains were given higher values than losses. This follows the psychology of prospect theory, which is the basis of behavioral finance and decision making with regard to saving and spending money.

Details of activation in different regions in terms of expectancies (prospects) and outcomes (winnings or losses) are shown in Table I below. As observed in Table I, multiple regions show differential patterns of signal change to good, bad and intermediate prospects. For example, one amygdala focus of activation in the left hemisphere of the brain, responds to the expectancy of a bad outcome while other regions such as GOb 5, 6, 8 only respond to good prospects. This data is the first to show lateralization differences for circuitry actually involved in reward function. In Table I, the column labeled ANOVA represents ANalysis Of Variance.

TABLE I

| | | Coordinates | | | Change from Baseline | | ANOVA | |
|---|---|---|---|---|---|---|---|---|
| Anatomy | ROI # | R/L | A/P | S/I | Prospects | Outcomes | Prospects | Outcomes |
| Frontal Lobe | | | | | | | | |
| GOb | 1 | −25 | 47 | −18 | B | 2, 8 | SP * TP | BI |
| GOb | 2 | 15 | 34 | −21 | G, I | 1 | — | — |
| GOb | 3 | −12 | 66 | −6 | — | — | — | — |
| GOb | 4 | 18 | 19 | −25 | — | 1, 9 | — | BI |
| GOb | 5 | 6 | 59 | −12 | G | 3 | — | BI |
| GOb | 6 | 25 | 59 | −18 | G | 2, 8 | — | BI * TP |
| GOb | 7 | −34 | 38 | −18 | B | 2 | — | — |
| GOb | 8 | −12 | 31 | −21 | G | 6 | — | BI |
| GOb | 9 | 28 | 44 | −12 | G, B | — | — | — |
| GOb | 10 | −25 | 13 | −9 | B | 2, 3, 7 | SP | BI, BI * TP |
| Temporal | | | | | | | | |
| Lobe | | | | | | | | |
| Medial | | | | | | | | |
| Amygdala | 11 | −18 | 3 | −15 | B | 5 | SP * TP | BI |
| Amygdala | 12 | 21 | −3 | −21 | — | 9 | — | BI |
| Subcortical | | | | | | | | |
| Gray | | | | | | | | |
| NAc | 13 | 12 | 16 | −6 | G, I, B | 1-3, 6, 7, 9 | SP | BI, BI * TP |
| SLEA | 14 | 18 | 0 | −6 | G, I, B | 1-3, 6-9 | SP | BI |
| Hypothalamus | 15 | 9 | −3 | −6 | G, I, B | 3, 6, 9 | SP, SP * TP | BI |
| Brainstem | | | | | | | | |
| VT | 16 | 12 | −18 | −12 | G, I, B | 3 | — | BI |

It has also been shown that the clustering of regions involved in expectancy and outcome assessment in different hemispheres of the brain exists. In particular, it is notable that there appears to be a right hemisphere predominance, for deep brain structures (e.g., NAc, SLEA) with regard to positive stimuli, while there is a left hemisphere dominance for negative stimuli in regions such as the amygdala and GOb. Data such as this show that right or left brain activation of reward circuitry may be important for defining salience of signal changes (i.e., valence or sign).

As noted above, many brain regions showing expectancy effects also show outcome effects. These matrices emphasize the different combinations of expectancy and outcome effects for these reward regions. For example, the SLEA can be observed to respond to median outcome effects in the context of intermediate expectancies and gains in the context of good expectancies. This pattern of activation, sets it apart from other reward regions; indeed, every region can be identified by its particular response patterns to rewarding or aversive stimuli (i.e., in this case, monetary losses).

Table II provides a summary of activation across multiple studies using different categories of reward. Table II shows that a common circuitry processes reward information, regardless of the category of the reward stimulus, whether drug, money or social stimulus. The observation that this is a generalized circuitry means that any type of object can be assessed regarding its rewarding properties to see how it falls along the continuum of reward (see FIGS. 3K, 3I regarding evaluating how it falls along the continuum of reward). Of further importance, the areas of brain activation that are common across these categories of reward were also observed to be activated during the perception of an aversive stimulus (see FIGS. 3E, 3F, and 3H, 3I). This commonality does not imply that all these regions work in the same way for rewarding and aversive stimuli. For example, negatively valenced signal is observed in the NAc to a painful stimulus, while positively valenced signal is observed in the NAc for a drug reward such as morphine.

Table II is divided into two main sections, one on expectancy, and one regarding outcomes. The left section on expectancy shows that across two studies with monetary reward and cocaine reward, expectancy effects lead to activation in a number of common areas, namely the GOb and bilateral NAc. These effects are different than the outcome effects in terms of signal intensity and waveform. Across a number of experiments—two with cocaine infusions, one with morphine, one with monetary reward, and one with a social reward (beautiful faces), common foci of activation were observed in the GOb, NAc, SLEA, and potentially the VT. The X's in the columns are superscripted to indicate more than one foci of activation in that region (i.e., X²=2 foci of activation, X³−3 foci of activation). Brackets around an X indicate that the statistical significance of the findings were just subthreshold for the experiment in question. It should be noted that there are two columns for the cocaine experiments, representing two completely separate cocaine experiments. The two columns for the beauty study represent positive vs. aversive outcomes. In this study, it was found that young men looking at beautiful male faces, devalued the images, indicating they were non-rewarding, while valuing the beautiful female faces, indicating that they, in contrast, were rewarding). It should be noted that the beauty experiment is not the only one with aversive and rewarding outcomes. For example a monetary reward experiment discussed below also had very explicit rewards vs. losses. The strongest results regarding aversive outcomes, though, are the pain studies, which show activation in the same GOb, NAc, and SLEA regions that are common across category of reward.

TABLE II

| Expectancy | | Monetary | Cocaine | Outcomes | | Cocaine | | | Monetary | Beauty | |
|---|---|---|---|---|---|---|---|---|---|---|---|
| Region | | Reward | Expectancy | Region | | (1) | (2) | Morphine | Reward | (+) | (−) |
| Gob | R | X² | X² | Gob | R | X | X | (X) | X³ | (X²) | |
| | L | X | X | | L | X | X | | X³ | | |
| NAC | R | X | X | Nac | R | X | X | X³ | X | X | (X) |
| | L | X | | | L | X | | X | | | X |
| SLEA | R | | | SLEA | R | (X) | X | X² | X | (X) | |
| | L | | | | L | X | | | | | X |
| Amygdala | R | | | Amygdala | R | (X) | X | | X | | |
| | L | X | | | L | X | | | X | | (X) |
| VT | R | | | VT | R | X | X | | X | | |
| | L | | | | L | X | X | X | | (X) | |

Referring now to FIG. 4, a noninvasive measurement apparatus and system for measuring indices of brain activity during motivational and emotional function is shown. In this particular example a magnetic resonance imaging (MRI) system 216 that may be programmed to non-invasively invasively aid in the determination of indices of brain activity during motivational and emotional function in accordance with the present invention is shown. Its should be appreciated however that other techniques including but not limited to fMRI, PET, IR, SPECT, CT, MRS, MEG and EEG may also be used to non-invasively measure indices of brain activity during motivational and emotional function.

MRI system 215 includes a magnet 216 having gradient coils 216a and RF coils 216b disposed thereabout in a particular manner to provide a magnet system 217. In response to control signals provided from a controller processor 218, a transmitter 219 provides a transmit signal to the RF coil 216b through an RF power amplifier 220. A gradient amplifier 221 provides a signal to the gradient coils 216a also in response to signals provided by the control processor 218.

The magnet system 217 is driven by the transmitter 219 and amplifiers 220, 221. The transmitter 219 generates a steady magnetic field and the gradient amplifier 221 provides a magnetic field gradient which may have an arbitrary direction. For generating a uniform, steady magnetic field required for MRI, the magnet system 217 may be provided having a resistance or superconducting coils and which are driven by a generator. The magnetic fields are generated in an examination or scanning space or region 222 in which the object to be examined is disposed. For example, if the object is a person or patient to be examined, the person or portion of the person to be examined is disposed in the region 222.

The transmitter/amplifier combination 219, 220 drives the coil 216b. After activation of the transmitter coil 16b, spin resonance signals are generated in the object situated in the examination space 222, which signals are detected and are applied to a receiver 223. Depending upon the measuring technique to be executed, the same coil can be used for the transmitter coil and the receiver coil or use can be made of separate coils for transmission and reception. The detected resonance signals are sampled, digitized in a digitizer 224. Digitizer 224 converts the analog signals to a stream of digital bits which represent the measured data and provides the bit stream to the control processor 218.

The control processor 218 processes the resonance signals measured so as to obtain an image of the excited part of the object. A display 226 coupled to the control processor 16 is provided for the display of the reconstructed image. The display 226 may be provided for example as a monitor, a terminal, such as a CRT or flat panel display.

A user provides scan and display operation commands and parameters to the control processor 218 through a scan interface 228 and a display operation interface 30 each of which provide means for a user to interface with and control the operating parameters of the MRI system 10 in a manner well known to those of ordinary skill in the art.

The control processor 218 also has coupled thereto a CNS signal processor 232, a correlation processor 234 and a data store 236. It should be appreciated that each of the components depicted in FIG. 4, except for the CNS signal processor 232 and the correlation processor 234 are standard equipment in commercially available magnetic resonance imaging systems.

It should also be appreciated that the MRI system must be capable of acquiring the data which can be used by CNS signal processor 232 and the correlation processor 234. In some embodiments, the CNS signal processor 232 and the correlation processor 234 may be provided as a general purpose processors or computers programmed in accordance with the techniques described herein to determine indices of brain activity during motivational and emotional function. For example, in some applications it may be desirable to provide a single processor or computer which is appropriately programmed to perform the functions of control processor 216, the CNS signal processor 232 and the correlation processor 234. In other embodiments, the CNS signal processor 232 and the correlation processor 234 may be provided as specially designed processors (e.g. digital signal processors) or other specially designed circuits. In any event the CNS signal processor 232 and the correlation processor 234 are unique in that they are programmed or otherwise designed to determine indices of brain activity during motivational and emotional function in accordance with the present invention as described herein.

The CNS signal processor 232 and the correlation processor 234 cooperate to determine indices of brain activity during motivational and emotional function. One particular technique for determining determine indices of brain activity during motivational and emotional function is described below in conjunction with FIGS. 5A-5C. Suffice it here to say that once CNS signals are obtained (e.g. via a non-invasive technique including but not limited to MRI, fMRI, PET, etc . . . ), the signals are localized to examine the function in a particular region of the brain. The particular manner in which such the signals are localized are dependent upon a variety of factors including but not limited to the technique or techniques (including equipment) used to extract the signals.

Once signals are extracted, the correlation processor 234 correlates empirical data with the measured signals. The correlation processor 234 then interprets the results of the correlation to a specific application The CNS signal processor 232 and the correlation processor 234 perform many of the functions described in phases 502-509 described below in conjunction with FIGS. 5A-5C which describe the Motivational/Emotional Mapping Process (MEMP) classification.

It should be appreciated that although processors 232, 234 are here shown a separate and distinct processors, in practice the functions described herein may involve the use of both processors 232, 234. Moreover, in practice all functions described herein as being performed by different processors (e.g. 218, 232, 234) may be performed by a single processor or by more than three processors. Thus, processors 232, 234 may cooperate as inter-digitated processors. Processor 232 may be involved in performing all or portions of Steps 502-507 (FIG. 5A) while processor 234 may be involved in performing all or portions of Steps 502, 503, 508a, 508b.

The remaining components of FIG. 4 perform the functions described in phase 501 of FIG. 5A and Step 518 of FIG. 5B.

Referring now to FIG. 5A, the general phases used in the Motivational/Emotion Mapping Process (MEMP) are illustrated. This process can be partially implemented using a CNS measurement system, such as system 14 described above in conjunction with FIG. 4. In a setup phase 500, the experimental paradigm is developed, subjects are screened and selected, and neuroimaging parameters are optimized.

In phase 501, brain imaging data is collected along with physiological and psychophysical data. Preferably the MRI system 14 of FIG. 4 is used to image the brain, however it should be appreciated that there are several other techniques known in the art to obtain brain imaging with sufficient resolution (approximately 5×5×5 mm) for the MEMP.

In a Signal Processing and Statistical Mapping of Imaging Data Phase 502, signal processing involves the normalization of data across subjects and experimental conditions, and transformation of data into a uniform space for averaging, or anatomically precise sampling of signals. Standard signal processing techniques of fMRI include, but are not limited to motion correction, signal intensity scaling, detrending, spatial filtering, temporal filtering, and morphing of the functional imaging data into a uniform space such as that of Talairach and Tournoux. Statistical mapping involves evaluating fMRI 3D data across time for significant changes relating to experimental conditions or any other variables such as subject physiology or psychophysical responses. Statistical evaluation involves some degree of location and scale estimation along with techniques for computing general effects and pairwise differences between experimental conditions.

In an Anatomic Localization Phase 503, anatomic templates for precise localization of fMRI signal changes are prepared. Anatomic scans, either acquired at the time of functional neuroimaging with the experiments or at another time, are transformed into the same uniform space as the functional brain data. For example, this may involve a Talairach transformation (i.e., brain anatomy from individuals is normalized into a standardized 3D reference system) cortical flattening. Alternatively the anatomic and functional data may be registered into the same coordinate system so that they have an aligned set of 3D axis and the anatomic data can be segmented and parcellated into precise anatomic locations for later superposition on the functional data. Segmentation and parcellation is a reproducible method using a standard format for locating and defining the boundaries of brain regions. The quantified volume of each brain region is one output of the process. Anatomic and functional data are ultimately co-registered so that fMRI functional data can be evaluated for each individual on their native anatomy. Such techniques may be the primary means of anatomic localization of significant signal changes, or be a supplement to use, of uniform anatomic spaces such as that of Talairach and Tournoux for primary anatomic analysis.

In a Hypothesis Testing and Determination of Significant Activity Phase 504, targeted anatomic regions having significant signal changes relating to experimental conditions, physiology, and psychophysical measures are evaluated. Experimental conditions include variables built into the experimental paradigm, variables built around the group or groups of subjects being scanned and potentially compared, variables involving any administered drugs or compounds, and variables involving repeated administration of are paradigm, or comparison of this paradigm to another paradigm. Hypothesis testing involves correction for the multiple comparisons between experimental conditions being made. Determination of significant activity throughout the entire brain, or throughout the entire set of acquired functional data, will also be performed using a correction for this larger set of comparisons. Hypothesis testing and determination of significant change will also be performed for comparisons generated by the physiology and psychophysics data.

In a Signal Evaluation Phase 506, signal features relative to the experiment are evaluated. Evaluation of signal features involves (1) determination of signal valence (i.e. sign); (2) intensity (i.e. magnitude or relative magnitude); (3) intensity over time (i.e. the waveform changes); and (4) adaptation dynamics (any adaptation of mean or median signal over time including habituation and sensitization processes).

This evaluation of signal features is important for understanding how a signal in a specified anatomic region may be significantly different between experimental conditions, or across physiological changes or changes in psychophysics responses. The evaluation of signal features is not limited to the four categories mentioned above. These four categories in particular, are mentioned because they allow us to evaluate patterns of signal within specified anatomic regions. These patterns within one anatomic region can also be compared to patterns within other anatomic regions. Sets of regions with similar signal features can then be “clumped” together for discussing the dynamics of activation across multiple brain regions.

In a Signal Quantification Phase 507, a calculation of specific indices which can be compared across experimental conditions across brain regions, and sometimes across separable experimental paradigms. The primary use of quantified indices of an fMRI signal is that sets of these indices become very precise descriptors of signal events in anatomic regions. These sets of indices (e.g., characteristics of the waveform such as the time-to-peak measure) can be used to categorize large numbers of brain regions by experimental condition. These categorizations of multiple regions quantify a “pattern” of activation which can be evaluated across multiple experimental conditions, or can be used to compare experimental condition effects to physiological effects or to psychophysics-relevant effects. These patterns can also be used to compare individual subjects, or follow them over time. Quantified signal indices compliment but do not replace the signal features described in Step 506 above.

In a Comparison of Experimental vs. Physiological Effects Phase 508a, patterns of significant signal change in hypothesized brain regions and elsewhere in the brain are compared and contrasted between experimental conditions and effects related to physiology. Similarly, signal features and quantified signal indices are compared and contrasted between experimental conditions and physiology. This is done to determine what experimental effects are truly independent of mainly global effects produced by body physiological changes.

In a Comparison of Experimental vs. Psychophysical Effects Phase 508b, patterns of significant change, signal features and quantified signal indices in hypothesized brain regions, and elsewhere in the brain are compared and contrasted between experimental conditions and effects associated with the psychophysical responses. This is done to determine which experimental condition effects and psychophysical response, effects are (dependently) linked, and which are independent.

In an Interpretation of Experimental Results Phase 509, experimental condition effects and psychophysical response effects which are independent and dependent on each other are evaluated with regard to known functions of the targeted (hypothesized) brain regions and other brain regions. Interpretation of experimental paradigm results in individual subjects or groups of subjects is performed against a background of established brain response features and quantified indices for particular paradigm conditions {a₁→aₙ}, which reflect (or were designed to interrogate) specific motivational or emotional functions. Thus, components of motivation function from blocks 80, 82, or 84 (in FIG. 2C), such as expectancy phase 86 through outcome phase 96, which reflect subfunctions of block 82, are connected to experimental paradigm conditions or psychophysical responses. This connection of experimental paradigm and psychophysics results to motivation and emotion functions is then used to answer the query leading to the initial formulation of the experiment.

Referring now to FIGS. 5B, 5C, the steps in the Motivational/Emotion Mapping Process (MEMP) are illustrated. The process begins as shown in Step 510 in which an experimental paradigm is developed targeting motivational/emotional function from one of the three general processes needed for motivated behavior. These processes are (1) determination of objectives for survival and optimization of fitness aversion; (2) extracting information from the environment regarding potential goal objects, events or internal states, of relevance to motivational function and meeting the above objectives; and (3) definition of behavior to obtain the goal objects and thus meet the objectives for survival. The experimental paradigm involves a number of discrete conditions which are to be independently measured or compared and are referred to as conditions {a₁→aₙ}. It is important to note that experimental conditions include variables built around the group or groups of subjects being scanned and potentially compared, variables involving any administered drugs or compounds, and variables involving repeated administration of one paradigm or comparison of this paradigm to another paradigm. The experimental paradigm may be integrated with parallel physiological measures (e.g., heart rate (HR), blood pressure (BP), Temperature, skin galvanic response SGR, etc.) and/or with parallel psychophysics measures (e.g., analog rating scales of pain or pleasure, response times etc.)

The types of experiments which can be developed in Step 510, can be quite diverse. Examples of experiments which can be split into conditions {a₁→aₙ} are provided by a representative cocaine vs. saline infusions study, and a monetary gain reward study, and a beauty bar-press procedure

For example in the cocaine vs. saline infusions, experiments were split into pre- vs. post-infusion conditions: namely, a₁=pre-cocaine infusion, a₂=post-cocaine infusion, a₃=pre-saline infusion, and a₄=post-saline infusion.

For the monetary experiment, there were nine experimental conditions depending on the combination of expectancy and outcome conditions for a wheel of fortune.

In the beauty bar-press procedure, subjects bar-press to keep a picture up longer, bar-press to get rid of a picture quicker, or do nothing. The time interval before each of these 3 conditions represents a₁, a₂, and a₃. These experiments result in a set of experimental conditions {a₁→aₙ} which are separable either in time, or by correlation with physiological or psychophysical measures.

Experiments developed in Step 510 incorporate principles from neurobiology, clinical pharmacology, cognitive neuroscience, decision theory, neurocomputation and medicine including psychiatry and neurology. The experiments are hypothesis driven. Regions can be specified a priori on the basis of the current neuroscience and medical literature at the time. Experiments incorporate a number of conditions whose comparison make it possible to attribute function to targeted brain regions. Examples of such experiments can be seen in double-blind cocaine infusions, thermal stimulation experiments to evaluate pain processing and monetary reward experiments (described below in more detail) Step 510 includes the development any off-line testing if required.

In Step 512, subjects are selected and screened for study. The subjects may be human, or animal, depending on the experimental question behind the experiment developed in Step 510.

In Step 514, neuroimaging parameters are optimized and tested. The optimized parameters are integrated into the experimental paradigm {a₁→aₙ}. The integration of any potential infusion with radioligand, nucleotide, or contrast material into the sequence of scans planned for experimental conditions {a₁→aₙ} occurs in Step 514.

A number of regions that can be targeted are subcortical grey matter structures. An attempt is made to reduce potential artifacts affecting signal from deep gray matter structures by optimizing machine parameters. For example, to see the nucleus accumbens or amygdala, one might acquire signal using nearly isotropic voxel dimensions and reduced echo times. In addition, shimming methods known in the art can be used to enhance the homogeneity of the mean magnetic field via use of second or higher order shims.

In Step 516, paradigm conditions {a₁→aₙ} are administered in temporal linkage with Step 518.

In Step 518, brain imaging results in signal acquisition in time and space using optimized machine parameters (including potential infusion with radioligand or contrast agent).

In Step 520, physiological and psychophysics parameters are measured in linkage with brain imaging from Step 518. Non-invasive physiological parameters (measured outside or inside the functional brain imaging unit) include any/all measure/s of physiological function such as heart rate (HR), blood pressure (BP) including systolic, diastolic and mean using a cuff, skin galvanic response (SGR), skin blood flow as measured by laser Doppler, respiratory rate (RR), electrocardiogram (EKG), pupilometry, electroencephalography (EEG) etc.

Invasive physiologic parameters can include blood pressure (via arterial line), blood oxygenation levels or any similar pulmonary measure using blood sampling, hormonal levels as measured by repeated blood sampling and subsequent assays, drug levels or levels of any injected compound which may be part of the experiment, etc.

Psychophysical parameters include any subjective response (which may be recorded by voice or a device (such as a mouse) used in the magnet by the subject to specific questions presented to them inside or outside the magnet. Examples include visual analogue scores, hedonic measures, reaction times, experiment guided responses (e.g., true/false), or other means of communicating internal states etc.

Note, most of the physiological parameters can be measured in animals and humans. However, psychological parameters are mostly specific to humans.

In Step 522, as an example of the many signal processing and statistical mapping techniques available for fMRI data, two basic approaches to fMRI data analysis will be described. In the first approach, the system targets a set of anatomically defined regions of interest (i.e., NAc, amygdala, SLEA, VT/PAG for a reward study), and evaluates signals from these regions using two statistical mapping techniques. A second approach evaluates signals throughout the entire brain, including the extended set of regions implicated in reward functions, such as the GOb, MPFc, CG, and Insula. This post-hoc analysis evaluates averaged data with a similar set of statistical methods as for targeted reward regions. The examination of the imaging signals, occurs in 3-D, relative to experimental paradigm. It should be appreciated that some of the MEMP Steps could become automated or semi-automated.

Prior to statistical mapping, initial signal processing involves motion correction which uses the automated image registration or some similar type of motion correction (AIR) algorithm or similar programs which are applied to individual data sets. After motion correction, all individual images are evaluated for residual motion artifacts. Functional MRI data may be intensity scaled and linearly detrended. Spatial filtering may be performed using a Hanning filter with a 1.5 voxel radius, and then mean signal intensity is removed on a voxel by voxel basis.

During analysis of the targeted reward regions, all individual structural and functional data sets are transformed into Talairach space to allow statistically significant findings to be aggregated across subjects. In contrast, for voxel-by-voxel analysis, whole brain structural and functional data are transformed into Talairach space prior to averaging across subjects. The averaged functional data is then statistically evaluated as described below in conjunction with Steps 522 through 566.

In parallel to the analysis of functional data using parametric statistical mapping (and multiple correlation mapping described below), as shown in Phases 502, 503 the structural scans for each individual have the targeted brain regions segmented (e.g., NAc, SLEA, amygdala, and VT). These segmentation volumes are then be transformed into the Talairach domain. Each activation cluster identified on the group average data is evaluated to determine its localization in these segmentation volumes. Each cluster, which is localized in a particular segmentation volume for 80% or more of the individuals comprising the average, is kept for subsequent analysis.

For the statistical parametic maps, these selected clusters in the targeted regions (e.g., NAc, SLEA, amygdala, and VT/PAG) are used to sample the individual Talairach-transformed functional data. This individual data are submitted for robust location and scale estimation using the Tukey bisquare method to evaluate experimental conditions and determine differences between them. Differences across experimental conditions may emerge quantitatively when conditions are sampled together (i.e., morphine vs. saline effects on thermal pain stimuli), or qualitatively in the form of differences in patterns of activation in each of the apriori structures when the conditions are sampled separately. For each analysis across conditions, clusters which have a significant result by robust analysis of variance (ANOVA) will then undergo pairwise contrasts.

As part of Step 526, individual fMRI data are also evaluated for correlational mapping of subjective effects (as from hedonic analog scales), and correlational mapping of physiological measures as correlational analysis will involve multiple correlation of both subjective ratings with the fMRI data set during which they were collected in each subject. Correlation maps are composed of correlation factors for each pixel. Correlation factors are transformed into probability values using a Fisher transformation. Correlation maps for each individual are anatomically morphed into the Talairach domain. These p-value maps are evaluated across each experimental group using a conjunction analysis to quantify the commonality of activations across experimental conditions. The conjunction maps representing the association of subjective effects with fMRI data in individuals are evaluated by identifying clusters of activation in the NAc, SLEA, amygdala, and VT.

Whole Brain Data (Voxel-by-voxel analysis of averaged data) is processed in Phases 502-504. Evaluation of brain areas not included in the initial set of targeted regions can involve use of whole brain data averaged across subjects. A number of statistical mapping procedures are currently available for post-hoc analysis. In one embodiment, a statistical mapping procedure is performed on a voxel-by-voxel basis, using both a Mean Field Theory (MFT) analysis, and a multiple correlation analysis.

Analysis of fMRI data can be broadly grouped as model-free or model-based methods, and time-preserving or non-time preserving methods. Most data analysis methods use distribution statistics, such as Student's t test or Kolmogorov-Smirnov statistics. In these designs a constant hemodynamic response during stimulation is assumed. These techniques are not time-preserving since they compare distribution of activated time points versus resting time points regardless of their time order. Model-based, time-preserving techniques, such as correlation analysis and in some cases, event-related fMRI maintain the temporal information by including in their analysis the particular time evolution of the model for the fMRI response. These techniques may have some limitations in detecting CNS activation if more than one hemodynamic response is present. The use of an a priori hemodynamic model may mask structures whose responses differ from the chosen model.

In Step 524, anatomical localization is performed using a number of different techniques. Preferably, anatomic localization is performed using universal anatomic coordinate systems (e.g., Talairach & Tournoux), individual anatomy (e.g., as with segmented brain volumes), and anatomically morphed anatomy (e.g., inflated flattened cortical surfaces).

Preferably, anatomically segmented and parcellated brain regions are used for anatomical localization of signal changes. It should be appreciated that alternate embodiments may be developed in the future for more sophisticated and detailed anatomical localization of signal changes observed with functional imaging.

The segmentation methodology, founded upon intensity contour and differential intensity contour concepts is used in Step 524. The cortical parcellation technique is based upon the concept of limiting sulci and planes and takes advantage of the observed relationships between cortical surface features and the location of functional cortical areas. An example set of operational definitions is presented in Caviness et al., 1996; Makris et al., 199 which is hereby incorporated herein by reference in its entirety. A critical advantage of this method is that definitions are unambiguously definable in a standardized fashion from the information visible in high resolution MRI.

As is known in the art, targeted regions (e.g., the NAc, SLEA, amygdala, VT/PAG) will have specific anatomic definitions. For instance, for the NAe, SLEA, anygdala, and VT/PAG, the following definitions can be used. The NAc is identified at the inferior junction between the head of caudate and the putamen. The NAc is delimited superiorly by a line connecting the inferior corner of the lateral ventricle and the inferior most point of the internal capsule abutting the NAc and laterally by a vertical line passing from the latter point. The VT/PAG and amygdala is directly visualized, and the posterior extent of amygdala is located at the identical coronal plane as the anterior tip of the anterior hippocampus. The PAG is contained in parcellation units that include the midbrain tegmentum. The SLEA region is identified anterioposteriorly from the midsection of the NAc extending back to the first substration nigra (SN) coronal section. It is identified medially by the hypothalamus (which extends anteroposteriorly from anterior commisure to include posteriorly the mammily body (MB), having a vertical line at the level of the optic tract or the lateralmost extent of the optic chiasm of the internal capsule as its lateral border and the interhemispheric midline as its medial border). All other anatomic regions are identified using both the Talairach coordinates of the max vox for each activation cluster in the average data, and their superposition with the averaged structural scans. In cases where there is disjunction between these two methods, activation is localized for each of the individuals comprising the average map, and tabulated as the percentage of individuals who contributed to the group image.

It should be appreciated that the signal processing and statistical analysis is described in terms of the current state of the art for fMRI data. Data collection techniques will likely change over the next few years. The statistical procedures will vary somewhat between neuroimaging techniques, but should all involve location and scale estimation, along with techniques for computing general effects and pairwise differences between experimental conditions. The inventive method is compatible with other imaging techniques and future imaging techniques which produce location and scale measurements having equivalent resolution characteristics to current fMRI imagers.

In Step 522, an examination of imaging signal, in 3-D, relative to experimental conditions {a₁→aₙ}, produces location and scale estimates for statistical evaluation of paradigm effects. The exact sequence of steps between Step 522 and Step 566, regarding statistical evaluation and anatomic localization may vary, as may the specific method for statistical evaluation or anatomic localization.

In Step 524, an anatomic framework or map in 3-D is generated which can localize fMRI signals.

In Step 526, examination of imaging signal, in 3-D, relative to physiology, and, separately relative to psychophysical function, producing location and scale estimates for statistical evaluation of physiology, & psychophysical effects on brain function.

In Step 528, images from Step 522 with those in 524 are merged to allow localization of brain imaging signal for experimental conditions {a₁→aₙ}.

In Step 530, brain imaging signals associated with physiology and psychophysics measures are localized.

During the Hypothesis Testing and Determination of Significant Activity Phase 504, brain impulse signal from targeted regions is identified on the basis of previous for reward/pain relevant regions, other imaging studies, or animal data.

The hypothesis testing and determination of significant activity shown in Phase 504, includes Steps 532-566. In Steps 532 and 534, thresholds of significance are computed for the statistical tests to allow for multiple statistical comparisons. This is done in a different fashion-depending on the type of statistical analysis being performed. One method involves using a region of interest analysis to sample maxima of signal change within targeted regions. The signal from these targeted regions in individuals is then submitted to an ANOVA analysis where the p value of threshold is corrected for the number of regions being sampled. In contrast to this, a voxel by voxel technique of analysis might incorporate another format of threshold correction. One means of doing this is to measure the volume of tissue sampled in targeted/hypothesized regions, to determine how many voxels cover this tissue, and to divide the p<0.05/x, where x=the number of voxels, to maintain an overall alpha level of less than 0.05. The volume of tissue for the entire brain is also then sampled and used in a similar fashion to produce a correction similar to a Bonferroni correction. After computing thresholds of significance for targeted and non-targeted regions, imaging data from targeted regions is marked.

In Step 532, an operator or an automated process splits localized results for experimental conditions {a₁→aₙ} into regions which are a priori (i.e., targeted) and those which are not.

In Step 534, an operator or an automated process splits localized results for physiology and psychophysical conditions to regions which are a priori (i.e., targeted) and those which are not.

Hypothesis testing continues in Steps 544-550. In Step 544, statistical threshold testing

based on Step 510 is performed on the targeted regions within the motivational & emotional circuitry.)

In Step 544, targeted brain regions are evaluated to determine if they have significant general effects and significant effects between experimental conditions. In Step 548, the same procedure is followed regarding the evaluation of physiologic and psychophysical effects in the fMRI data.

In Step 546, evaluation of whole brain data (i.e., this may be on a voxel by voxel basis for every voxel acquired during the experiment in the brain), is performed to determine if there are significant general effects and effects between conditions. In Step 550, the same procedure as in 546 is followed, to evaluate physiological and psychophysical effects. The output of the process in 544 is noted as Step 552 and 554, the output of Step 546 is noted as Step 556 and 558, the output of 548 is noted as Step 560 and 562, and the output of 550 is noted as 564 and 566. The rationale for segregating these outputs in this fashion, is that only 552 and 556 contribute the input to the processing in 568. Similarly, only the output of Step 560 and Step 564 contribute the input to the processing of Step 570.

In Step 552, significant activity in targeted regions from threshold testing in Step 544 is determined. In Step 554, subthreshold activity in targeted regions from threshold testing in Step 544 is determined. In Step 556, significant activity in non-targeted regions from threshold testing in Step 546 is determined. In Step 558, subthreshold activity in non-targeted regions from threshold testing in Step 546 is determined. In Step 560 significant activity in targeted regions from threshold testing in Step 548 is determined. In Step 562, subthreshold activity in targeted regions from threshold testing in Step 548 is determined. In Step 564, significant activity in non-targeted regions from threshold testing in Step 550 is determined. In Step 566, subthreshold activity in non-targeted regions from threshold testing in Step 560 is determined.

In Step 568, the system evaluates of signal features relative to the experiment (valence, graded intensity information intensity over time or wave/are, and adaptation dynamics. Two examples of evaluating signal features with biological significance are described below. In particular, the use of valence information (from pain and facial expression stimuli), and graded intensity information (from monetary reward stimuli) are described.

In Step 568, during fMRI of rewarding or aversive stimuli in humans, positive activation (signal change) in the NAc following rewarding stimuli (including monetary reward, beauty, and drug reward) and a negative activation (decreased signal change) following noxious thermal stimuli is observed. These findings directly show that painful stimuli are assessed distinctly from rewarding stimuli, as reflected by an altered valence of NAc signal change. In Step 570, the system evaluates of signal features relative to subjective ratings (intensity over time).

In Steps 572 and 574, the signals are quantified and compared between experimental conditions. In Step 572, the signal features within the same anatomic foci and between different anatomic foci are quantified (i.e., to produce for instance, time to peak and dispersion measures) and compared to experimental conditions {a₁→aₙ}. Also in Steps 572 and 574, the use of quantified signal indices can describe signal events in anatomic regions. These anatomic regions can then be categorized by these descriptions to show a pattern of signal response across many regions. For example, thermal pain data can be evaluated to produce time-to-peak measures (Tₚ) and dispersion measures (Δ) (i.e. the width of the signal change in response to a painful stimulus from the point of inflection of the signal to its return to baseline). These Tₚ and Δ measures were then evaluated across all regions showing significant signal change (both targeted/hypothesized regions, along with all other brain areas) and divided on the basis of being above or below the mean Tₚ and mean Δ. This division was legitimized since there were two peaks of Tₚ and Δ across the set of regions with significant change. The categorization of regions into a matrix with (a) Tₚ<mean and Δ<mean, (b) Tₚ<mean and Δ>mean, (c) Tₚ>mean and Δ<mean, and (d) Tₚ>mean and Δ>mean, categorizes the entire set of anatomic regions activated by the experimental condition of applying an aversive (painful) thermal stimulus. This pattern of activated regions can be directly compared to the patterns from other experimental conditions to determine differences between conditions in terms of anatomic regions involved in the different conditions. The categorization of Tₚ and Δ above was compared to that from a non-aversive/non-painful thermal stimulus to show the differences in brain regions processing these two categories of stimulus. There are many potentially quantifiable signal indices. Depending on the number of indices used, an N-dimensional matrix can be used to categorize the regional activations so characterized with the N indices.

In Step 574, the signal features within the same anatomic foci and between different anatomic foci are quantified and compared to physiological and psychophysical measurements. In Step 576, the overlap between experimental condition and physiological effects, and the overlap between experimental conditions and psychophysical effects is evaluated. In Step 578, experimental conditions which cannot be segregated from physiological conditions are identified. These regions do not receive any more processing. In Step 580, experimental conditions which can be segregated from physiological conditions in the same anatomic foci, and between different ones are identified. In Step 582, experimental conditions which cannot be segregated from psychophysical effects in the same anatomic foci, or between different ones are identified. In Step 584, experimental conditions which can be segregated from psychophysical effects in the same anatomic foci, or between different ones are identified. In Step 582 or Step 584 the subject can be either conscious or non-conscious.

One example of the steps in Phase 506 would be a comparison of cocaine infusion maps generated by the comparison of the pre-infusion interval with the post-infusion interval with the statistical maps generated by correlation of subjective ratings with the brain signal. Thus, activations produced by the cross-correlation of rush and/or craving ratings with brain signal can be overlaid with the activations which represent the response to cocaine in general. Some activations from the general cocaine map will correspond with the activations that correlate to rush, others will correspond with the activations that correlate to craving, while a third set may correspond to both, and a fourth set may not correlate to either craving nor rush.

In Step 586 offline studies (done outside neuroimaging system) or questionnaires can optionally be used to modulate interpretation of imaging data. Performance on offline studies or scores from offline questionnaires can be correlated with quantitative signal measures from the functional imaging process.

In Step 588, the system interprets the results from the experiment in terms of motivational and emotional function, or changes therein. Signal features in specific anatomic regions or between different anatomic regions convey a specific picture or script of motivation/emotion function. The biological signals define the motivational and emotional function effected by the experimental paradigm.

In Phases 502-504 statistical analysis is performed on hypothesized/targeted regions (e.g., NAc, SLEA, VT/PAG, Amygdala). Parametric statistical mapping of experimental effects in individual fMRI data begins with an aggregation process, i.e., all experimental runs for an individual are concatenated. Individual data for the aggregated experiments is then transformed into the Talairach domain. Data common to each experiment is then averaged across all individuals. This averaged functional data then undergoes a statistical comparison of its baseline condition vs. all categorically common experimental conditions, to produce the masks used to collect signal intensity data from individual subjects. Thus, for each experimental condition, a t-test is performed between a common baseline and all time-points for all experimental conditions which may be subsequently compared. From these statistical comparisons, clusters of activation are identified using a cluster-growing algorithm. To maintain an overall alpha<0.05, this algorithm will localize activation meeting a corrected threshold of p<0.05/x, (i.e., P for the max vox) where x is the number of hypothesized brain regions interrogated. The cluster growing algorithm will select voxels with p<0.05/x in a 7 mm radius of a voxel with a minimum p-value (i.e., max vox). Max vox peaks are within a cluster of at least 3 voxels, each of which meets the statistical threshold. Max vox peaks will also be separated by at least 4 mm from any other putative peak.

As shown in Phases 502-504, the MFT approach avoids such issues by determining statistical significance using cross correlation of each pixel with a mean hemodynamic response (MHR). The MHR is obtained for a subset of active pixels found active by using a T-test. The MFT approach has been used for a noxious heat experiment, and has been found to yield more information than standard approaches, including more robust levels of significance for signal changes, increased numbers of brain regions that are observed to be activated, and temporal differences in signal time courses for proximate activations (e.g. early activation in putative reward regions and late activation in classic pain regions).

Also in Phases 502-504, in conjunction with the MFT analysis, a multiple correlation analysis of the averaged whole brain data using averaged subjective ratings is performed. For both the MFT and multiple correlation analysis, significance is determined by applying a Bonferroni like correction for multiple comparisons. Correction levels are determined as follows:

(1) for apriori regions the corrected p value is 0.05 divided by nₐₚᵣᵢₒᵣᵢ (apriori=number of pixels sampled in the apriori regions)

(2) for post hoc regions, the p value is 0.05 divided by n_{post hoc} (posthoc=number of pixels in whole brain grey matter region sampled)

As shown in Phases 502-506, assessment of aversive stimuli as distinct from rewarding stimuli also involves the pattern of reward circuitry activation, as shown by distinct patterns of reward region activity seen during studies of the visual processing of negative facial expressions. In these studies with facial expressions, studies with facial expressions which are responses to aversive stimuli, or conditions, positive left amygdala activation during the visual processing of fearful faces and positive signal change of the right amygdala following presentation of sad faces is observed.

Experiments can be explicitly designed to dissect the sub-functions of the informational system for motivated behavior. For instance, in one experiment, monetary reward in a game of chance resembling gambling at a slot machine is used to dissect out activity in reward regions related to the evaluation of probability information (i.e., expectancy), and valuation information (in this case under the general outcome Phase of the system. This monetary reward experiment represents the first demonstration that circuitry involved in human motivation can be dissected into sub-component functions. An important feature of the ability to dissect sub-functions of the informational system for motivated behavior is ordered activation in sets of targeted reward regions which reflect the relative magnitude of the reward can be observed. Observing the NAc, SLEA, hypothalamus, and amygdala, can determine how rewarding stimuli are relative to each other.

Time course verification of statistical maps occurs in Phases 506 and 507. Foci of apparent significant change in hypothesized regions, and elsewhere in the brain, are further evaluated by examining the corresponding signal intensity vs. time curves, both for time course data taken from ROI constrained activation clusters (in individuals), and for post-hoc voxel focused activation maps. This also provides a means of determining an estimate of mean signal change and confirming that regional activation coincides with the timing of stimulus presentation.

Referring now to FIG. 6, a chart shows the relationship between motivation and neuroscience and molecular biology and genetics 602. Oval shaped reference lines 610-618 indicate that relationships exist between each of the measurement categories cognitive neuroscience (behavior) 600, human neuroimaging (distributed neural ensembles) 604, animal neuroimaging 606, electrophysiology (cells, neural ensembles) 608 and molecular biology and genetics at molecular and gene level 602. FIG. 6 is a diagram illustrating an association between functional neuroimaging in humans and animals. The importance of functional neuroimaging in humans and animals is apparent when considering that it is the primary means by which gene and molecular function can be linked to their behavioral effects.

FIG. 6 describes a working format for the interaction of a number of basic neuroscience techniques that measure brain/neuronal signals from various spatial scales. Thus for example, molecular biology and genetic studies predominantly work with animals to define the contribution of specific genes, modification of these genes or gene products (e.g., receptors) and the effects of moleculues (e.g., neurotransmitters) on neuronal function. This evaluation is performed at a cellular/molecular level. However, such techniques may use neuronal markers of activity (for example c-fos) to determine the function of groups of neurons throughout the neuraxis. However, this measure is made in-vitro (i.e., special staining methods of tissue harvested from animals). Electrophysiology on the other hand may measure the response of a single or multiple neurons to specific activation/perturbation (which may be sensory, electrical or chemical). Groups of neurons within the CNS may therefore show patterns of response indicative of a particular function of a neuron, group of neurons or brain region. Neuroimaging, animal or human, allows for the evaluation of signals from neuronal circuits in the living condition. Lastly, cognitive neuroscience and other experimental psychological disciplines allow a description of behavior that can be quantified and interdigitated with neuroimaging (e.g., monetary reward paradigm, using data from prospect theory).

Several experiments specific to motivation and emotion function have been performed using the techniques described above. These experiments have produced specific information regarding motivation/emotion functions. For instance, these experiments have involved graded responses to monetary reward in a game of chance, bar press experiments indicating a preference to various stimuli, and experiments evolving direct aversive/rewarding sensations.

In one experiment, the principles of prospect theory (as that term is understood in experimental psychology and behavioral) were incorporated into a game of chance with money to evaluate normative reward circuitry function during functional magnetic resonance imaging (fMRI) at 3 Tesla. The paradigm involved a sequence of single trials with spinners that shared a subset of outcomes, and segregated expectancy from monetary loss or gain.

In Step 512, twenty right-handed male subjects were recruited for this experiment, of which eight subsequently were shown after the experiment to have uncorrectable motion or spiking artifact, leading to twelve usable data sets. All subjects were medically, neurologically, and psychologically normal by self-report and review of systems.

This experiment was performed to map the hemodynamic changes that anticipate and accompany monetary losses and gains under varying conditions of controlled expectation and counterfactual comparison. The paradigm developed in Step 510 involved subjects viewing stimuli projected onto a mirror within the bore of the magnet, while maintaining a stable head position by means of an individually molded bite bar. The display consisted of either a fixation point or one of 3 disks (“spinners”). Each spinner was divided into 3 equal sectors. The “good” spinner could yield either a large gain (+$10), a small gain (+$2.50), or no gain ($0), the “bad” spinner could yield a large loss (−$6), a smaller loss (−$1.50), or no loss ($0), and the “intermediate” spinner could yield a small gain (+$2.50), a small loss (−$1.50), or neither a loss nor a gain ($0). Providing larger gains than losses was implemented to compensate for the tendency of subjects to assign greater weight to a loss than to a gain of equal magnitude (per project theory).

Before the game began, subjects were shown each spinner 3 times so as to learn its composition. Each trial consisted of (1) a “prospect phase,” when a spinner was presented and an arrow spun around it, and (2) an “outcome” phase, when the arrow landed on one sector and the corresponding amount was added to or subtracted from the subject's winnings. During the prospect phase, the image of one of the 3 spinners was projected for 6 sec, and the subject pressed one of three buttons to identify the displayed spinner, thus providing a measure of vigilance. The display was static for the first 0.5 sec, and then a superimposed arrow would begin to rotate. The arrow would come to a halt at 6 sec, marking the end of the prospect phase. During the first 5.5 sec of the ensuing outcome phase, the sector where the arrow had come to rest would flash, indicating the outcome. A black disk was then projected as a visual mask during the last 0.5 sec of the 12-sec trial. On fixation-point trials, an asterisk would appear in the center of the display for 15.5 sec, followed by the 0.5-sec mask.

The pseudo-random trial sequence was fully counter-balanced to the first order so that trials of a given type (spinner+outcome) were both preceded and followed once by all 9 spinner/outcome combinations and 3 times by fixation-point trials. Thus, the average 1-trial “history” and “future” was the same for trials of every type. Eight runs with 19 trials apiece were presented to subjects. Only results of the last 18 trials were scored for each run, since the initial trial was inserted into the run sequence purely to maintain counter-balancing. Runs were separated by 2-4 min rest periods. The same trial sequence was used for all subjects, generating winnings of $142.50, to which was added the $50 endowment. At the end of the scanning session, subjects completed a questionnaire rating their subjective experience of each spinner and outcome using an 11-point opponent scale.

The timing of stimulus events in this experiment, and the rationale for the data analysis, were based on two fundamental assumptions. A first assumption was that the hemodynamic-control system is approximately linear in the brain regions targeted by this experiment, on the basis of results from conditions tested to date. A second assumption, was that, given appropriate counterbalancing, the compound response could be “peeled apart” by means of selective averaging and comparison of impulse-like hemodynamic responses.

Subject instructions were developed in Step 510 and administered in Step 516. Using a set text, subjects were informed that they would be participating in a series of games of chance. At the start of these games, they would receive an endowment of $50 to cover possible losses, and informed of the maximum they could win over the course of the experiment. In the unlikely event that they lost more then their endowment, they would receive no money, but would receive a picture of their brain in action and have a clinical scan on record, worth approximately $1600. For each game of chance they would see a wheel of chance with three sectors. The wheel would move for some time, and the spinner would eventually land on one of the sectors, determining how much they received for that particular game. There would be three wheels of chance, which differ in their general level of outcomes, and would be termed the bad, medium, and good spinners. Subjects were informed they would see each of these spinners in a short video to acquaint them with the game. They were further informed to identify each spinner shown for each game as rapidly as possible using a button box, and to refrain from speech during the scan. After reading the instruction text, subjects' questions were answered, and they then observed a brief set of 10 trials (including the fixation trial) to familiarize them with the stimuli.

Physiological & psychophysical measures of behavior were monitored in Step 520. Subjects made behavioral responses throughout the study, consisting of identification of each spinner as it was presented. Subjects identified spinners using a button box, with the first key on the left (index finger) being used to identify the bad spinner, the second key on the left (middle finger) being used to identify the medium spinner, and the third key on the left (ring finger) being used to identify the good spinner.

In Step 518, subjects were scanned on an instascan device (3 T General Electric Signa; modified by Advanced NMR Systems, Wilmington, Mass.) using a GE head coil. Imaging for all experiments started with a sagittal localizer scan (conventional T1-weighted spoiled gradient refocused gradient echo (SPGR) sequence; through-plane resolution=2.8 mm; 60 slices) to orient, for subsequent scans, the slices to be acquired for functional scanning. This scan was also used as the structural scan for Talairach transformation. Next, an automated shimming technique was used to optimize B₀ homogeneity. Radio-frequency full-width half-maximum (FWHM) line-width after shimming of primary and secondary shims produced a measure of 32.4±2.2 for the 12 subjects with motion-correctable functional data. After shimming, experimental slices were prescribed, with 18 slices parallel to the AC-PC line and covering the NAc, amygdala, basal forebrain, and VT. In this orientation, an SPGR T1-weighted flow-compensated scan was obtained (resolution=1.6 mm×1.6 mm×3 mm), primarily to aid Talairach transformation during data analysis (see Breiter et al, 1996a). The fourth scan was a T1-weighted echo planar inversion recovery sequence (TI=1200 msec, in-plane resolution=1.57 mm) for high-resolution structural images to be used in preliminary statistical maps (but not with Talairach transformed or averaged maps). Finally, functional scans involved a T2*-weighted gradient echo sequence (TR=2 s, TE=35 ms; Flip=70°; in plane resolution=3.1×3.1 mm, through-plane resolution=3 mm, FOV=40×20 cm; 18 contiguous slices, images per slice=108 per run). The shortened TE and nearly isotropic voxel dimensions had been optimized previously in Step 514 to minimize imaging artifacts in the regions of interest.

Post-paradigm subjective relays were collected in Step 516. After finishing paradigm, subjects completed a questionnaire regarding cumulative gains, and their experience of the prospect and outcome phases of the experimental trials as a means of determining whether they experienced the monetary task in the manner predicted by prospect theory. The questionnaire specifically queried subjects' ability to follow cumulative gains/losses during the experiment, estimates of total winnings, and their subjective experience of spinner presentation, plus outcome from each spinner. To make these ratings of each spinner, and each outcome on the three spinners, subjects marked their response on an 11-point opponent scale ranging from very bad (−5) to very good (+5). Subjects were subsequently informed of their total gains from the experiment. In this particular study, no further offline or neuropsychological measures unrelated to the paradigm itself were performed as in Step 586.

Data Analysis on behavioral data collected during the paradigm was performed in Step 526. The integer output for each behavioral rating was checked against the trial sequence, and performance was listed for each individual. The mean ±standard error of the mean (SEM) were computed across the 12 subjects with motion-correctable functional data for each of the eight runs to ascertain that response errors were <5% per subject.

Data Analysis on post-paradigm data was performed in Step 526. The real-number responses of subjects with motion-correctable functional data were tabulated and evaluated using robust methods paralleling those detailed for the fMRI data (see Statistical Mapping, ROI-based Analysis (Steps, 522-566). Specifically, for the subjective ratings of spinner a statistical expert system performed an analysis of raw residuals and recommended against use of variance-adjusted weights and the Tukey bisquare estimator. The efficiency of the robust (bisquare) analysis was only 85% as great as the efficiency of the traditional least-squares approach, so the recommendation of the expert system was accepted, and a least-squares components ANOVA (one-way) performed with subsequent pairwise comparisons.

For the subjective ratings of outcomes, boxplots of the residuals indicated a number of potential outliers, the presence of which were confirmed with an analysis of raw residuals form the robust fit. The efficiency of the robust (bisquare) analysis was greater than the efficiency of the least squares approach as confirmed with a normal probability plot of residuals using student zed residuals, and hence the expert system recommended use of variance-adjusted means and the Tukey bisquare estimator. This recommendation was accepted, and a bisquare components ANOVA (two way—bins nested in spinner) performed with subsequent pairwise contrasts.

FMRI data was processed in Phases 502, 504. Signal processing of fMRI blood oxygen level dependency (BOLD) Data before Statistical Mapping occurred in Step 522. To reduce head motion, each subject was positioned using a bitebar, and BOLD data was motion corrected using a motion correction algorithm. After motion correction, time-series data were inspected to assure that no data set evidenced residual motion in the form of cortical rim or ventricular artifacts>1 voxel. From this analysis, 8 of 20 subjects were found to have uncorrectable motion or spiking artifact, leaving a final cohort of 12 subjects for further evaluation. Motion correction (mean ±SEM) of the BOLD data revealed an average maximal displacement for each of the eight runs of 0.43±0.097 mm, 0.67±0.16 mm, 0.72±0.18 mm, 0.71±0.15 mm, 0.80±0.19 mm, 1.16±0.30 mm, 1.33±0.39 mm, 1.47±0.43 mm. Motion displacement led to corrections for movement, in terms of the mean correction per time point for each of these runs, of 0.22+0.04 mm, 0.49±0.13 mm, 0.56±0.15 mm, 0.55±0.11 mm, 0.65±0.16 mm, 1.00±0.29 mm, 1.19±0.37 mm, 1.29±0.41 mm.

Step 522 for all eight runs, fMRI data in the Talairach domain was normalized by intensity scaling on a voxel-by-voxel basis to a standard value of 1000, so that all mean baseline raw magnetic resonance signals were equal corresponding to Step 522). This data was then detrended to remove any linear drift over the course of scan. Spatial filtering was performed using a Hanning filter with 1.5 voxel radius (this approximates a 0.7 voxel gaussian filter). Lastly, mean signal intensity was removed on a voxel-by-voxel basis.

In Step 522 trials were selectively averaged. In total, there were 10 trial types (spinner+outcome), including the fixation baseline. Prospect and outcome phases of the trials each lasted 6 seconds. Given the standard delay of 2 seconds for the onset of the hemodynamic response to neural activity, at least 14 seconds of BOLD response needed to be sampled for selective averaging across trial type. Six seconds of pre-stimulus sampling were incorporated for use in subsequent data analysis as a baseline to zero the onset of each trial. This is a common practice in evoked response experimentation. Counterbalancing was performed to the first order, so that the 6 seconds before the onset of each trial, when averaged across all iterations of that trial, would represent a common baseline against which to normalized the onset of each trial. Accordingly, selective averaging was performed for 20 second epochs.

Each individual's set of infusion images, along with the associated conventional structural scans, were transformed into Talairach space and resliced in the coronal orientation with isotropic voxel dimensions (x,y,z=3.125 mm). (Steps 522, 524 in FIG. 5). Optimized fit between functional data and structural scans was then obtained via translation of exterior contours.

Talairach-transformed structural and functional data (i.e., the selectively averaged trials, N=10) were averaged across the 12 subjects with interpretable BOLD data (Steps 522, 524 FIG. 5).

Statistical mapping, ROI-based analysis and statistical mapping of main effects as ROI's was performed as shown in Phases 502-504 above. All time-points collected during the prospect phase of the experiment, and all time-points collected during the outcome phase of the experiment were statistically evaluated by correlation analysis with a theoretical impulse function. The impulse function for the predicted hemodynamic response was generated using a gamma function. To eliminate cross-trial hemodynamic overlap, the correlation maps were generated with the difference between all prospect data and fixation epoch data, and with the difference between all outcome data and fixation epoch data using time-point by time-point comparison.

Subsequently, clusters of activation were identified using a cluster-growing algorithm. In order to maintain an overall α<0.05, this algorithm specifically localized activation which met a corrected p-value threshold of p<0.007 for the number of hypothesized brain regions being interrogated. Regions of interest (ROI)s were delineated by the voxels with p<0.007 in a 7 mm radius of the voxel with the minimum p-value (i.e., max vox). Max vox peaks had to be within a cluster of at least 3 voxels, making the statistical threshold, and separated by at least 4 mm from any other putative max vox peak. ROIs identified in this manner were then used to sample the individual prospect data (N=10 ROIs) and outcome data (N=6 ROIs).

During the Anatomic Localization Phase 502, in Steps 522, 524 and 526, statistical maps of group averaged data were superimposed over high-resolution conventional T₁-weighted images which had been transformed into the Talairach domain and averaged. Primary anatomic localization of activation foci was performed by Talairach coordinates of the maximum voxel from each activation cluster (see section on determination of activation clusters), with secondary confirmation of this via inspection of the juxtaposition of statistical maps with these coronally resliced T1-weighted structural scans. Confirmation of subcortical localization of activations followed the region of interest conventions described previously for the NAc (previously referred to as the NAc/SCC and here referred to as the NAc due to greater spatial resolution), SLEA (previously referred to as the basal forebrain or BF), amygdala, and VT. The GOb ROI conventions were not previously described, and are here delineated. Namely, the GOb (BA 11/47) was identified anteriorly behind the ventral surface of the frontal pole (BA10). It began with the orbital gyri (anterior, lateral, and medial) which are visible by the beginning of the orbital sulci, and extended posteriorly to the beginning of the SLEA of the basal forebrain which is visible by the extinguishing of the orbital sulci (transverse orbital sulcus). Laterally, the GOb extended to the anterior horizontal ramus of the Sylvian fissure, and medially, it extended to the olfactory sulcus.

As shown in Phases 502-504 priori regions evaluated for activation clusters included the NAc, amygdala, and VT (for prospects), and the SLEA, amygdala, hypothalamus, and GOb (for outcomes). Regions hypothesized for one condition (i.e., prospects or outcomes), were also evaluated for the other. In total, 10 clusters of signal change were noted for these a priori regions during the prospect phase of the experiment. Six other clusters of signal change were noted in a priori regions during the outcome phase of the experiment.

Signal time-course analysis of ROI's was performed in phases 502-504. The normalized fMRI signal was averaged, at each time point, within each activation cluster falling within an ROI. As described above, the averaged data were assembled into time courses, 20 sec in duration, which included a 6-sec epoch prior to trial onset.

An exploratory analysis of the time courses was performed in order to examine the across-subject distribution of the averaged fMRI signal in each cluster. First, the signals for each subject were transformed into deviations from the across-subject mean at each time point within each trial type. The deviation scores for the period beginning 2 sec following trial onset and ending 2 sec following the end of the trial were selected for exploratory analysis; this segment was used because it contained the data that were later used for hypothesis testing concerning expectancy and outcome responses. The deviation scores within the selected time period were combined across time points and trial types and displayed as a normal probability (“quantile-quantile”) plot. If the scores of the subjects were distributed normally, such a plot would be a straight line passing through the origin, with a slope equal to the standard deviation. Normal probability plots of data from some clusters did not deviate strongly from linearity, suggesting that the signals recorded from the different subjects were distributed in an approximately normal fashion. In contrast, substantial deviations from linearity, consistent with the properties of contaminated normal distributions, were noted in the case of several clusters. Thus, it was decided to employ robust statistical methods to describe the time courses. Such statistics are less subject than conventional parametric statistics to the influence of extreme values (“outliers”) and provide more efficient estimates of the central tendency (“location”) and dispersion (“scale”) of contaminated normal distributions. As described below, a formal test of the relative efficiency of the conventional and robust measures was carried out in order to determine whether robust or conventional least-square statistics were the most appropriate for hypothesis testing.

The robust estimates of location and scale are based on the Tukey bisquare estimator (phases 502-504). This estimator weights scores as a function of their deviation from the sample median. The weights decline smoothly to zero in a bell-shaped fashion as the deviation from the median grows. To compute the location estimate, each score is first expressed as a scaled deviation from the sample median:

$${u}_{i} = \frac{{x}_{i} - M}{c ⨯ MAD}$$

where

xᵢ=fMRI signal for subject i at a given time point

M=median of the fMRI signals for all subjects at that time point

c=a tuning constant and

MAD=the median of the absolute deviations from the median

The weighting function is
wᵢ=(1−uᵢ²)² if |uᵢ|≦1; wᵢ=0 if |uᵢ|>1,
the robust estimate of location (T_{bi}) is

$${T}_{bi} = M + \frac{\sum ( ( {x}_{i} - M ) ⨯ {w}_{i} )}{\sum {w}_{i}} ,$$

and the robust estimate of scale (s_{bi}) is

$${s}_{bi} = \frac{{n}^{\frac{1}{2}} ⨯ ( \sum {( {( {x}_{i} - M )}^{2} ⨯ {( 1 - {u}_{i}^{2} )}^{4} )}^{\frac{1}{2}} )}{\vert \sum {w}_{i}^{\frac{1}{2}} ⨯ ( 1 - 5 {u}_{i}^{2} ) \vert}$$

where n=the number of subjects

The turning constant, c, determines the point at which the weighting function reaches zero. As the value of this constant grows, progressively fewer data points receive zero weight, and the location estimate approaches the mean; as the value of this constant shrinks, progressively fewer data points are rejected, and the location estimate approaches the median. A tuning constant of 6 was employed to compute the location and scale estimates used to graph the signal time courses and their confidence intervals. Given normally distributed data, such a tuning constant would result in assignment of a zero weight to all observations falling more than 4 standard deviations from the median. In the case of the observed distributions, the median percentage of data points assigned a weight of zero was 1.24%. The range for 15 of the 16 clusters was 0.47-2.16%, whereas the percentage of data points rejected in the case of the remaining cluster was 5.86%.

A Baseline adjustment was made. The robust estimates of location and scale were computed first from untransformed data. A within-subject subtraction procedure was then used to align the signal time courses for each trial type with a common baseline. As shown in FIG. 7A, in the case of the data to be used for analysis of expectancy responses, the subtrahend consisted of the median fMRI signal during the six seconds prior to trial onset plus the first two seconds of the trial. (Due to the delay in the hemodynamic response, the signal during the first two seconds of the trial should reflect neural activation prior to trial onset.) This median value was then subtracted from the fMRI signals obtained during the subsequent 12 seconds. In the case of the data to be used for analysis of outcome responses, the subtrahend consisted of the median fMRI signal during the first six seconds of the trial (the prospect phase) plus the first two seconds following presentation of the outcome. Thus, in both cases, the median of the signals recorded during the preceding epoch was subtracted from the signals from a given trial phase.

Following the application of the subtraction procedure, new robust estimates of location and scale were computed. FIG. 7B illustrates the effect of the subtraction procedure on the robust estimates of location and scale; all data are from a cluster centered in the NAc (12, 16, −6). The solid vertical line denotes trial onset, and the dashed vertical line denotes the time at which the outcome is revealed. Thus, the expectancy phase ends, and the outcome phase begins, at the dashed vertical line; due to the delay in the hemodynamic response, the data points lying on each vertical line likely reflect events during the preceding epoch.

The robust estimates of location and scale were used to compute the 95% confidence intervals. Due to the fact that the average weight is less than one, the degrees of freedom must be corrected accordingly. The number of degrees of freedom were multiplied by 0.7 in constructing confidence intervals about the robust estimates of location. The expression for the confidence interval is

$${T}_{bi} \pm ( {t}_{( 0.7 ⨯ ( n - 1 ) )} ⨯ \frac{{s}_{bi}}{\sqrt{n}} )$$

In the a Hypothesis Testing and Determination of Significant Activity Phase 504, tests for differences between time courses were carried out using a statistical expert system such as RS/Explore. It should be appreciated that there are several methods and expert systems which can perform the statistical analysis. Separate analyses of the transformed data for the expectancy and outcome phases were conducted.

The multiple-regression module of RS/Explore was employed to carry out an analysis of variance (ANOVA) as part of Steps 544-550. In the cases of 12 of the 16 clusters, the data selected for this analysis consisted of the transformed fMRI signals during the period beginning 2 sec following trial onset and ending 8 sec following trial onset. This period lags the timing of the expectancy phase of the trial by 2 sec, consistent with other reports of hemodynamic delay post experimental stimulation. Examination of the time courses for these 12 clusters confirmed that signals whose confidence intervals cleared zero did indeed lag the onset of the trial by 2 sec. However, in the case of the remaining clusters, the lag was longer. For example, the peak signal in cluster GOb(6) occurred at 6 sec, and that the signal was still elevated at 8 sec. In the four cases such as this one, signal epochs selected for statistical analysis matched the time interval during which the peak signal was attained, and the maximum signal under the curve was observed. Thus, for cluster GOb(6), a 4 second lag allowed selection of the time interval with both the peak signal and maximum signal under the curve.

The data segment selected for analysis of expectancy responses in the case of the 3 other ROIs also consisted of the points at 4, 6, and 8 seconds. Regardless of the hemodynamic lag, the duration of the sampled period was 6 seconds.

The dependent variable in the expectancy ANOVA was the transformed BOLD signal, and the predictors were the spinner and time point. Both spinner and time point were defined as categorical (non-continuous) variables, thus forcing the analysis software to carry out an ANOVA in lieu of fitting a regression surface. By defining the independent variables in this fashion, it was possible to avoid making assumptions about the form of the time courses.

At the outset of the analysis, the statistical expect system compared the relative efficiencies of the Tukey bisquare estimator and conventional least-square statistics. In the cases of 15 of the 16 clusters, the Tukey bisquare estimator was found to be more efficient and thus, a robust ANOVA was carried out; graphical confirmation of the need for a robust estimator was provided by normal probability plots. In the remaining case, the least-squares estimator was found to be slightly (˜1%) more efficient and thus, as recommended by the expert system, conventional least-square methods were employed.

A second test carried out prior to the ANOVA compared the within-cell variances. In 15 of 16 clusters, these were found to be sufficiently similar that the use of variance-adjusted weights was not recommended. However, in the remaining cluster, the differences between the within-cell variances were sufficiently large as to cause the expert system to recommend the use of variance-adjusted weights.

The results of primary interest in the expectancy ANOVA were the main effect of spinner and the spinner×time point interaction. A main effect of spinner indicates a difference in the magnitude of the fMRI signals corresponding to the presentation of the three spinners; a spinner×time point interaction indicates the form of the signal time courses differed across spinners. Given that ANOVAs were carried out on the signals from 16 different clusters, a more stringent alpha level (0.003) was used than the conventional 0.05 value as the threshold for a significant effect.

In cases that met the criterion alpha level, the pair-wise across-spinner contrasts were computed at each of the three time points. Regardless of whether the main effect of spinner or the spinner×time point interaction met the significance criterion, the confidence band surrounding the location estimate was compared to zero. As in the case of the data from the interviews. Given that multiple comparisons were carried out, simultaneous confidence intervals reflecting the variance at all time points during the expectancy phase were used in this comparison.

The outcome-phase ANOVA was largely analogous to the expectancy-phase ANOVA. In all cases, the data employed fell within a 6-sec period beginning 2 sec after the onset of the outcome phase. The BOLD signal served as the dependent variable, and spinner, trial type, and time point served as the predictors; trial type, a categorical variable, was nested within spinner. (A $10 win following the presentation of the good spinner constitutes one trial type, whereas a $2.50 win constitutes another.)

Prior to the ANOVA, the expert system was used to determine whether robust or least-square statistics were more efficient and whether the use of variance-adjusted weights was recommended. A robust ANOVA was carried out in the case of 13 clusters, and a conventional least-square analysis was carried out in the remaining 3 clusters. Variance-adjusted weights were used in 7 of the 16 clusters. In all cases, the recommendations of the statistical expert system were accepted.

The results of primary interest in the outcome ANOVA were the main effect of trial type and the trial type×time point interaction. A main effect of trial type indicates a difference in the magnitude of the fMRI signals corresponding to the presentation of the different within-spinner outcomes; a trial type×time point interaction indicates that the form of the signal time course varied across trial type. As in the case of the expectancy-phase ANOVAs, the criterion alpha level was set to 0.003.

In cases that met the criterion alpha level, pair-wise contrasts were computed between the three trial types within each spinner, at each of the three time points. Regardless of whether the main effect of trial type or the trial type×time point interaction met the significance criterion, the confidence band surrounding the location estimate was compared to zero. (refer to relevant table) As in the case of the data from the expectancy phase, simultaneous confidence intervals were used in this comparison.

In Steps 522 and 524 as part of the Statistical Mapping of Imaging Data phase 502 data was produced for the Post-hoc voxel-by-voxel correlational analysis in Steps 546 and 550. This analysis sought to determine if regions not included in the hypotheses were potentially active during either the prospect/expectancy phase of the experiment, or the outcome phase. Toward this end, statistical correlational maps were generated against a theoretical impulse (i.e., gamma) function. Specific paired comparisons for the prospect and outcome data were the same as the post-hoc comparisons after the ANOVA analysis. These paired comparisons were all performed against the medium prospect or the intermediate outcome with one exception, namely all comparisons between the good and bad spinners, or the high and low outcomes, were deemed to be redundant since their main comparison was already contained in the dyadic comparisons of good to intermediate, and bad to intermediate spinners.

Clusters of positive and negative signal change were identified for each paired comparison using the automated cluster growing algorithm described above. In order to maintain an overall α<0.05, this algorithm specifically localized activation which met a corrected p-value threshold for the volume of tissue sampled in the a priori regions (i.e., of p<1.48×10⁻⁴ for prospects, and p<4.96×10⁻⁵ for outcomes). All other regions had to meet a corrected (Bonferroni) threshold for significance of p<7.1×10⁻⁶ for the estimated volume of brain tissue per subject sampled in this experiment. As previously, max vox peaks identified by the cluster growing algorithm had to be within a cluster of at least three voxels, of which the two voxels which were not the peak had to meet the statistical threshold of p<0.07 and be within a 7 mm radius of the max vox.

All activations were further checked against the functional image data to ascertain that they did not overlap areas of susceptibility artifact. Such overlap was determined by whether or not a voxel's signal intensity during the baseline was less than the average voxel in its slice by 50% of the difference between the average voxel signal intensity in the slice and the average voxel signal intensity outside of the slice.

In Phase 506 significant differential responses to monetary outcomes were recorded from the NAc, SLEA, and hypothalamus to the three outcomes on the good spinner ($10.00, $2.50, $0.00). For these ROIs, the time courses diverged similarly, with signal declines during the $0.00 outcome, and less marked declines in the case of the $2.50 outcome. The highest signal levels were recorded in response to the highest value ($10.00) outcome, and in the NAc and SLEA, the outcome phase response to this outcome rises towards the end of the trial. In these ROIs, the value of the normalized BOLD signal during the outcome phase tracks the subjects' winnings.

The outcome-phase time courses were aligned to a common baseline by subtracting the median of the normalized BOLD signals recorded during the prospect phase. Thus, even in the absence of a hemodynamic response to the outcome, the recorded signal may have decreased during the outcome phase simply due to the waning of the prospect response. The key to distinguishing bona fide responses to the outcomes from the decaying phase of preceding prospect responses is the differential nature of the outcome-phase responses. As shown by the significant effect of outcome or the outcome by time point interaction in the ANOVAs carried out in 12 of the 16 ROIs, differential outcome-phase responses were indeed observed, distinguishing these outcome results from those of the preceding prospect phase. Nonetheless, the decay of prospect-phase responses may have contributed to driving the outcome-phase signals below zero, which was the case at 37 of the 49 time points at which the outcome-phase signals differed reliably from the baseline. Thirty of these 37 time points moving below zero belong to the NAc, SLEA, and hypothalamus alone. In contrast to these subcortical signals, 11 of the 12 time points that move reliably above the baseline belong to GOb ROIs.

The dominant pattern in the most sustained outcome-phase responses (those that cleared the baseline reliably at the greatest number of time points) is typified by the signals recorded from the NAc, SLEA, and hypothalamus. For these three ROIs, relative to the median of the prospect-phase responses, the signal at the end of the outcome phase is lowest in response to the worst outcome on the good spinner ($0.00), somewhat higher in response to the small gain ($2.50), and highest in response to the large gain ($10.00).

A strikingly different pattern is observed in the case of cluster GOb(4). In that case, the responses to the two most extreme outcomes ($10.00, −$6.00) are higher than the responses to the other outcomes on the respective spinners. Thus, the responses in this ROI provide information about the magnitude of the outcome but not about its sign. Only one other time course, the response to the worst outcome on the bad spinner (−$6.00) in the right amygdala, deviates reliably from the baseline at more than one outcome-phase time point. Again, it is the response to an extreme outcome that stands out.

In phase 507, a number of prospect responses demonstrated signals with distinct time to peak measures. Signals from subcortical and brainstem structures with robust simultaneous 95% confidence bands that cleared the baseline, peaked at 4 seconds in 10 of 13 cases. Several of the signals that peaked later were recorded in GOb ROIs, for instance, differential lags are apparent during responses to the good spinner in the SLEA and in GOb(6). It is important to note, for the SLEA and GOb(6), that slice acquisition occurred in interleaved fashion in the axial domain, parallel to the AC-PC line, with a through-plane resolution of 3 mm. The functional data from activations in the SLEA (Talairach coordinates: 18, 0, −6) and GOb(6) (Talairach coordinates: 25, 59, −18) were acquired only one slice apart. Thus, at each time point, at most 100 msec separated acquisition of signal in the SLEA and GOb(6). In contrast, the peak of SLEA signal leads the peak of the GOb(6) signal by 2 seconds, and the GOb(6) response remains near its peak value for an additional 2 seconds during which time, the SLEA signal declines. The temporal separation of these acquisitions cannot be accounted for by technical or averaging constraints.

Phase 508, was not applicable to this experiment.

Research on the psychology of monetary gains and losses shows that the subjective response to an outcome depends on the alternative outcomes available and on prior expectation. In Phase 509, the interpretation of the results suggest that this was also the case in the BOLD signals recorded in the NAc, SLEA, and hypothalamus in response to the $0 outcomes. On the good spinner, $0 is the worst of the three outcomes available. The responses to this outcome fall throughout the outcome phase, dropping below the other time courses. In contrast, the NAc and SLEA responses to the $0 outcome on the bad spinner are rising at the end of the outcome phase, around the time when a hemodynamic response to an outcome might be expected to peak; these signals climb above the responses to the $0 outcome on the good spinner, as does the bad-spinner response in the Hyp. The $0 outcome on the bad spinner is the best available on that spinner. Indeed, the form of the BOLD time courses recorded during the outcome phase of bad-spinner trials on which the outcome was $0 resembles the form of the responses in the NAc and SLEA to the best outcome ($10.00) on good-spinner trials. Finally, the psychological research predicts that the $0 outcome on the intermediate spinner, which falls between the two other values, will be experienced as near-neutral. The normalized BOLD time courses corresponding to presentation of this outcome (small circles) fluctuate near the zero baseline.

The design of this experiment takes into account several principles that have emerged from the psychological study of judgment and decision. Paramount among these is the view that the emotional impact of an outcomes depends strongly on the context within which they are experienced. Thus, the experiment was designed so as to control and manipulate prior expectations as well as post-hoc comparisons with the alternative (“counterfactual”) outcomes available. Both the psychological and neurobiological literature suggest that different processes are brought to bear when anticipating and experiencing outcomes. Thus, the trials were structured so as to separate over time the responses of the subjects to prospects and outcomes. Psychological research shows that losses with respect to a neutral point tend to loom larger than gains of the same magnitude. Larger gains than losses were employed in an attempt to offset this tendency. Five different monetary amounts were used, enabling us to determine how the BOLD signal varied as a function of the magnitude and sign of the outcomes. By including one common outcome on all three spinners, the influence of expectation and counterfactual comparison could be assessed. The asset position (cumulative winnings) of the subject was not displayed, thus increasing the likelihood that performance on each trial would be referenced to a common baseline. Modeling of the design of the present study on principles well established in prior psychological research on judgment and decision may have been crucial to the clarity and orderliness of the BOLD signals as well as to their tight linkage to trial events.

All references cited herein are hereby incorporated herein by reference in their entirety.

Having described preferred embodiments of the invention, it will now become apparent to one of ordinary skill in the art that other embodiments incorporating their concepts may be used. It is felt herefore that these embodiments should not be limited to disclosed embodiments, but rather should be limited only by the spirit and scope of the appended claims.

What is claimed is:

1-26. (canceled)

27. A method for diagnosing and/or treating a subject with a brain functional disorder, the method comprising:
(a) exposing a subject to an experiment intended to elicit from the subject at least one of a motivational and an emotional state;
(b) exposing the subject to an impulse function;
(c) in response to the subject being exposed to the experiment, identifying clusters in brain regions of a subject in which a response is measured;
(d) analyzing a time course of signal change in each of the clusters in each of the brain regions of interest; and
(e) identifying deviations from normal patterns of motivational circuitry function to diagnose a brain functional illness.

28. The method of claim 27, wherein identifying clusters in step (c) is based on the presence of overall hemodynamic changes in brain regions linked to prospect and outcome phases of an experiment, averaged over both trial types and subjects.

29-34. (canceled)

35. A method of providing a non-invasive marker for a brain functional illness, comprising:
(a) non-invasively obtaining signals of activity from anatomic and functional brain regions that mediate emotion or motivation or both during central nervous system function;
(b) mapping the signals to each of the anatomic and functional brain regions to generate a pattern of activity; and
(c) correlating the pattern to the brain functional illness to produce the non-invasive marker of the brain functional illness, wherein the brain functional illness is selected from the group consisting of schizophrenia, psychosis, hedonic deficit syndromes, anxiety disorders, and attention deficit disorder.

36. The method of claim 35, wherein the anatomic and functional brain regions are selected from the group consisting of the orbital gyrus (GOb), ventral tegmentum/periaqueductal gray (VT/PAG), nucleus accumbens (NAc), sublenticular extended amygdale (SLEA), cingulate gyrus, primary somatosensory cortex (S1), secondary somatosensory cortex (S2), thalamus, insula, cerebellum, prefrontal cortex, amygdala, hypothalamus, parahippocampal gyrus, hippocampus, entorrhinal cortex, ventral pallidum, dorsal striatum, primary motor cortices (M1), secondary motor cortices, supplementary motor cortex (SMA), frontal eye field (FEF), rostral ventromedial medulla (RVM), and brainstem subnuclei.

37. A method of diagnosing a brain functional illness in a subject, comprising:
(a) providing a first pattern of central nervous system (CNS) activity associated with normal function;
(b) measuring a second pattern of CNS activity in the subject, wherein said measuring comprises:
(i) non-invasively obtaining signals of activity from at least two different regions of the central nervous system; and
(ii) quantifying and localizing signals to their specific regions of origin to generate the second pattern of activity; and
(c) comparing the first and second patterns to diagnose the brain functional illness.

38. The method of claim 37, wherein the brain functional illness is selected from the group consisting of schizophrenia, psychosis, depression, hedonic deficit syndromes, anxiety disorders, attention deficit disorder, drug addiction, and obsessive compulsive disorder.

39. The method of claim 37, wherein the at least two different regions are selected from the group consisting of the orbital gyrus (GOb), ventral tegmentum/periaqueductal gray (VT/PAG), nucleus accumbens (NAc), sublenticular extended amygdale (SLEA), cingulate gyrus, primary somatosensory cortex (S1), secondary somatosensory cortex (S2), thalamus, insula, cerebellum, prefrontal cortex, amygdala, hypothalamus, parahippocampal gyrus, hippocampus, entorrhinal cortex, ventral pallidum, dorsal striatum, primary motor cortices (M1), secondary motor cortices, supplementary motor cortex (SMA), frontal eye field (FEF), rostral ventromedial medulla (RVM), and brainstem subnuclei.

40. A method of monitoring the progression of a brain functional illness excluding obsessive compulsive disorder and drug addiction in a subject comprising:
measuring a pattern of CNS activity in selected CNS regions in said subject over time, said measuring comprising:
(i) non-invasively obtaining signals of activity from at least two different regions of the central nervous system; and
(ii) quantifying and localizing signals to their specific regions of origin to generate the pattern of activity;
wherein the pattern of activity as a function of time is indicative of the progression of the brain functional illness.

41. The method of claim 40 wherein said brain functional illness is selected from the group consisting of schizophrenia, psychosis, depression, hedonic deficit syndromes, anxiety disorders, and attention deficit disorder.

42. The method of claim 40, wherein the at least two different regions are selected from the group consisting of the orbital gyrus (GOb), ventral tegmentum/periaqueductal gray (VT/PAG), nucleus accumbens (NAc), sublenticular extended amygdale (SLEA), cingulate gyrus, primary somatosensory cortex (S1), secondary somatosensory cortex (S2), thalamus, insula, cerebellum, prefrontal cortex, amygdala, hypothalamus, parahippocampal gyrus, hippocampus, entorrhinal cortex, ventral pallidum, dorsal striatum, primary motor cortices (M1), secondary motor cortices, supplementary motor cortex (SMA), frontal eye field (FEF), rostral ventromedial medulla (RVM), and brainstem subnuclei.

43. (canceled)

44. The method of claim 27, wherein said brain functional illness is selected from the group consisting of schizophrenia, psychosis, depression, hedonic deficit syndromes, anxiety disorders, and attention deficit disorder.

45-46. (canceled)

47. The method of claim 27, further comprising determining brain functional illness prognosis.

48. The method of claim 27, further comprising planning brain functional illness treatment.

49. The method of claim 27, further comprising monitoring progression of a brain functional illness excluding obsessive compulsive disorder and drug addiction.
